(19) United States
(12) Patent Application Publication
(10) Pub. No.: US 2024/0280457 A1
(43) Pub. Date: Aug. 22, 2024

(54) AIRBORNE VIRUS SENSORS

(71) Applicant: University of Utah Research Foundation, Salt Lake City, UT (US)

(72) Inventor: Massood Tabib-Azar, Salt Lake City, UT (US)

(21) Appl. No.: 18/569,195

(22) PCT Filed: Jun. 9, 2022
(86) PCT No.: PCT/US2022/032824

§ 371 Date: Dec. 11, 2023

Related U.S. Application Data
(60) Provisional application No. 63/208,695, filed on Jun. 9, 2021.
(60) Provisional application No. 63/210,914, filed on Jun. 15, 2021.

(51) Int. Cl. G01N 15/02; G01N 1/22; G01N 33/53; G01N 33/569

(52) U.S. Cl. CPC G01N 15/02; G01N 1/2205; G01N 33/5308; G01N 33/56983; G01N 2333/165; G01N 2469/10

(57) ABSTRACT

Technology is disclosed related to devices, systems. and methods for detecting a target particle. The device can include a field generator source which emits an incident electromagnetic field: a resonator having a focusing structure to focus the incident electromagnetic field in a gap region that accepts the target particle; and a receiver to detect a resonant signal from the resonator, where the resonant signal shifts due to presence of the target particle in the gap region.

### RELATED APPLICATION

This application claims priority to U.S. Provisional Application No. 63/208,695 filed Jun. 9, 2021 and U.S. Provisional Application No. 63/210,914 filed Jun. 15, 2021, which are each incorporated herein by reference.

### GOVERNMENT INTEREST

This invention was made with government support under grant no. 1931100 awarded by the National Science Foundation. The government has certain rights in this invention.

### BACKGROUND

The recent pandemic motivated the development of sensors to detect airborne viral particles over large areas. Microwave remote sensing techniques have been extensively developed during the past few decades and can offer good sensitivity to changes in the impedance of objects interrogated through free space. Respiratory viruses such as SARS-CoV-2 can become airborne and can spread through aerosolized particulates generated by coughing or talking. Detection in air or on surfaces can provide information used to monitor, control and prevent widespread transmission.

The aerosolized exhaled particles are mostly composed of human saliva that may also attract other airborne particles such as soot and dust particles. A healthy person's saliva has an average relative permittivity of about 76, while water has a relative permittivity of 78 at room temperature and 80 at 20° C. Human saliva is about 98-99% water and 1-2% of biomarkers, urea, ions, enzymes, and other components. Human saliva exhibits different electrical characteristics in the same person depending on his or her hydration level, fasting, activity level, age, and other conditions. Saliva also contains viruses and bacteria in infected individuals. SARS-COV-2 infected individuals can have large load of viruses in their saliva approaching around one million perml. SARS-COV-2 is a 70-125 nm spherical virus with spiking proteins S1 and S2.

### SUMMARY

In one embodiment, an airborne particle sensor for detecting a target particle can comprise: a field generator source, a resonator, and a receiver. In one aspect, the field generator source can be configured to emit an incident electromagnetic field. In another aspect, the resonator can have a focusing structure configured to focus the incident electromagnetic field in a gap region that is configured to accept the target particle. In yet another aspect, the receiver can be configured to detect a resonant signal from the resonator. The resonant signal can shift due to the presence of the target particle in the gap region.

In another embodiment, a method can be provided for sensing airborne particles. In one aspect, the method can comprise emitting an incident electromagnetic field from a source. In another aspect, the method can comprise focusing the incident electromagnetic field into a gap region of a resonator that is configured to accept a target particle. In yet another aspect, the method can comprise detecting a resonant signal from the resonator.

In yet another embodiment, an airborne particle sensing system can comprise a source, an array of airborne particle sensors, and a receiver. In one aspect, the source can be configured to emit an incident electromagnetic field. In another aspect, the array of airborne particle sensors can be housed in a ventilated structure. In yet another aspect, each airborne particle sensor can comprise a resonator having a focusing structure configured to focus the incident electromagnetic field in a gap region that is configured to accept a target particle. In another aspect, the receiver can be configured to detect the resonant signals from the array of airborne particle sensors.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

Features and advantages of the disclosure will be apparent from the detailed description which follows, taken in conjunction with the accompanying drawings, which together illustrate, by way of example, features of the disclosure.

FIG. 1 is a schematic of the experimental set up used to perform X-band and terahertz studies to detect airborne target particles in accordance with an example.

FIGS. 2A-21 are representative illustrations of several metamaterial configurations in accordance with an example.

FIG. 3 is an equivalent circuit to model the interaction of a sample with Rₛ resistance and Cₛ capacitance with through gap region of MTM sensor in accordance with an example. The interfacial resistance and capacitance (Rᵢ and Cᵢ) electrically model the MTM-sample interaction.

FIG. 4 is an illustration of the experimental setup used for measurements in accordance with an example.

FIG. 5A is an illustration of a spherical sensor array including a microwave antenna in accordance with an example.

FIG. 5B is an illustration of a two-dimensional sensor array including a microwave antenna in accordance with an example.

FIG. 6A is an illustration of a sensor structure in accordance with an example.

FIG. 6B is an illustration of a funnel structure in accordance with another example.

FIG. 7 depicts a method for sensing airborne particles in accordance with an example.

FIG. 8A is a S21 spectra of the Fabry-Perot resonator with single atomic layer graphene sensor and its frequency shift with 5 μL of uninfected and infected saliva in accordance with an example. The viral density was 1000 viruses/μL of the infected saliva.

FIG. 8B is graph of Frequency shift (f₀-f₀′ where f₀ is the resonant frequency without the saliva while f₀′ is the resonant frequency with the saliva) of the 16.4 GHZ Fabry-Perot paper filter resonator as a function of saliva volume in accordance with an example.

FIG. 8C is a S21 spectra of 5 μL infected and un-infected saliva using the single layer graphene between 0.75 and 0.83 Terahertz in accordance with an example. The main deviation between the two normalized spectra is shown by the red arrow. The curves were normalized to the S21 spectrum of the plain graphene.

FIGS. 9A-9D are graphs of electrical properties of a tested metamaterial based particle sensor in accordance with an example.

FIGS. 10A-10C is an illustration of E-fields at 10 GHz at different phase angles in accordance with an example. FIG. 10A illustrates a 0° phase angle: FIG. 10A illustrates a 90° phase angle; and FIG. 10C illustrates a 180° phase angle. The incident field was vertically polarized.

FIGS. 11A-11C is an illustration of simulated E-field components in the spoke structure in accordance with an example. FIG. 11A is an illustration of the X components of the E-field: FIG. 11B is an illustration of the Y components of the E-field: FIG. 11C is an illustration of the combined E-field. The incident E was vertical (Y).

FIG. 12A is a graph of the S11 spectra for 1 μL water and saliva sample placed in the gap in accordance with an example. FIG. 12B is a graph of the frequency spread at resonance for 1 μL of water and saliva samples in accordance with an example.

FIG. 13A is a graph of the S11 spectra of an MTM sensor with 1 μL water, uninfected saliva, and SARS-COV-2 infected saliva in accordance with an example. FIG. 13B is a graph of the resonance frequency of an MTM sensor with 1 μL SARS-COV-2 infected saliva, uninfected saliva, and water droplets in accordance with an example.

FIG. 14A is a graph of the S11 spectra for dielectric samples in accordance with an example. FIG. 14B is a graph of the resonance frequency as a function of duroid samples with different sizes in accordance with an example.

FIG. 15A is a graph of the S11 spectra for iron particles in accordance with an example. FIG. 15B is a graph of the resonance frequency as a function of iron size in accordance with an example.

FIG. 16 is a graph of the S11 spectra for various samples placed on a dielectric substrate with εᵣ=3.43 in accordance with an example.

FIG. 17 is a graph of the S11 spectra for 4 MTM sensor arrays with and without 1 μL deionized water droplets in accordance with an example.

These drawings are provided to illustrate various aspects of the invention and are not intended to be limiting of the scope in terms of dimensions, materials, configurations, arrangements, or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a channel” includes reference to one or more of such channels and reference to “the electrode” refers to one or more of such electrodes.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a “target particle” can be any type of particle that is desired to be detected using a airborne particle detector. In one example, the particle can be a pathogen (including a virus, bacteria, fungus, or protist), a metal, a dielectric, the like, or combinations thereof.

As used herein, a “focusing structure” is a structure that concentrates an electromagnetic field at a specific location. In one example, the focusing structure can be a metamaterial structure that can concentrate an electromagnetic field of a selected frequency at a specific location.

As used herein, a “gap region” is a location of concentrated electromagnetic field in an airborne particle sensor that can receive a target particle.

As used herein, a “2D layer” is a crystalline solid consisting of a single layer of atoms. In one example, a 2D layer can include one or more of: graphene, tungsten disulfide, molybdenum disulfide, graphyne, borophene, germanene, silicene, stanene, plumbene, phospherene, antimonene, bismuthene, 2D platinum, 2D palladium, 2D rhodium, a 2D alloy, graphane, boron nitride nanosheet, titanate nanosheet, borocarbonitrides, MXenes, transition metal dichalcogenide monolayers, 2D silica, niobium bromide, niobium chloride, germanane, Ni₃(HITP)₂, other semiconductors, the like, or combinations thereof.

As used herein, comparative terms such as “increased,” “decreased,” “better,” “worse,” “higher,” “lower,” “enhanced,” “improved,” “maximized,” “minimized,” and the like refer to a property of a device, component, composition, or activity that is measurably different from other devices, components, compositions, or activities that are in a surrounding or adjacent area, that are similarly situated, that are in a single device or composition or in multiple comparable devices or compositions, that are in a group or class, that are in multiple groups or classes, or as compared to an original or baseline state, or the known state of the art. For example, an FPGA with “reduced” runtime can refer to an FPGA which has a lower runtime duration than one or more other FPGAs. A number of factors can cause such reduced runtime, including materials, configurations, architecture, connections, etc.

Reference in this specification may be made to devices, structures, systems, or methods that provide “improved” performance. It is to be understood that unless otherwise stated, such “improvement” is a measure of a benefit obtained based on a comparison to devices, structures, systems or methods in the prior art. Furthermore, it is to be understood that the degree of improved performance may vary between disclosed embodiments and that no equality or consistency in the amount, degree, or realization of improved performance is to be assumed as universally applicable.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein.

The term “coupled,” as used herein, is defined as directly or indirectly connected in a biological, chemical, mechanical, electrical, or nonelectrical manner. “Directly coupled” structures or elements are in contact with one another. In this written description, recitation of “coupled” or “connected” provides express support for “directly coupled” or “directly connected” and vice versa. Objects described herein as being “adjacent to” each other may be in physical contact with each other, in close proximity to each other, or in the same general region or area as each other, as appropriate for the context in which the phrase is used.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Occurrences of the phrase “in one embodiment,” or “in one aspect,” herein do not necessarily all refer to the same embodiment or aspect. Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### Example Embodiments

An initial overview of invention embodiments is provided below, and specific embodiments are then described in further detail. This initial summary is intended to aid readers in understanding the technological concepts more quickly but is not intended to identify key or essential features thereof, nor is it intended to limit the scope of the claimed subject matter.

Viruses are composed of capsid proteins and glycoprotein and inner DNA or RNA cores that determine their dielectric properties. Most RNA and DNA viruses have dry relative permittivity of 8-10. This means that when a saliva sample (having relative permittivity of 78) is infected with SARS-COV-2 virus, depending on its viral load, it can have a slightly lower dielectric constant (e.g., a relatively permittivity less than 78) compared to uninfected saliva. SARS-COV-2 viral particles are negatively charged in the saliva thus providing a slightly higher ionic conductivity.

A real-time (1-5 mins) virus detection system can be used to reduce the spread of infection. Large numbers of rapid sensors have been developed including: saliva-based GO-decorated Au/FBG SARS-COV-2 sensors, facile biosensors, paper-based electrochemical biosensor, nano-materials based biosensors, colloidal particles and unique interfaces-based SARS-COV-2 detection magnetic nano-sensor, electrochemical saliva sampling, rapid electronic SARS-COV-2 sensors, plasmonic sensors, and the like. There is also a THz sensor which can detect spike proteins of the SARS-COV-2. However, most of these sensors involve sample preparation and use at least a long time for providing results. The sensors disclosed herein can detect the virus in shorter times.

In one example, a technology is described for a microwave detection-based technology to passively screen the air in public spaces for SARS-COV-2 and other pathogens, thereby providing an early warning system for potential community spread or super spreader events. In one example, a conductive mesh or filter can be used to trap particles in the air, some of which may contain pathogens. The mesh or filter can be modified to trap specific pathogens or particles through appropriate functionalization such as by using aptamers.

Microwaves can be used to detect the presence of pathogens, which can alter the conductivity of the mesh, to allow for the sampling of a large volume of air and a threshold sensitivity. Saliva samples containing virus (having a concentration of about 1000 virions/μL) can alter the conductance of the mesh filter in a detectable way relative to samples without saliva and samples having uninfected saliva. The results demonstrate that viral load in saliva can have a detectable contribution to microwave and terahertz absorption. The terahertz absorption characteristics can be used as its signature for viral detection.

In the X band, the signature on graphene and/or a metallization pattern can provide a signature for viral detection. For example, an X-band, free-space microwave sensor having 30 radial spokes connected in a central hub with a gap region can be used for viral detection. The sensor structure can have an electric dipole at 10 GHz with a split circular disc capacitor at the center. Viruses, dust, and soot particles in the gap region can change the sensor's impedance and its reflection coefficient monitored by a horn antenna and a network analyzer. The sensor sensitivity was 85.02 MHz/μL for deionized water, 89.5 MHz/μL for uninfected saliva, and 94.6 MHz/μL for SARS-COV-2 infected saliva with 103 viruses/μL. Its sensitivity: (i) for a dielectric sample εₚ˜5.84) was 3.23 MHz/mm³, and (ii) for iron particles was 16.25 MHz/mm³. When the samples are smaller than λ/30 at 30 GHZ, the samples could not be detected on uniform dielectric or metallic substrates without the spoke structure. A 2×2 array of spoke sensors was also constructed and tested as a feasibility study for designing larger metamaterial (MTM) periodic arrays.

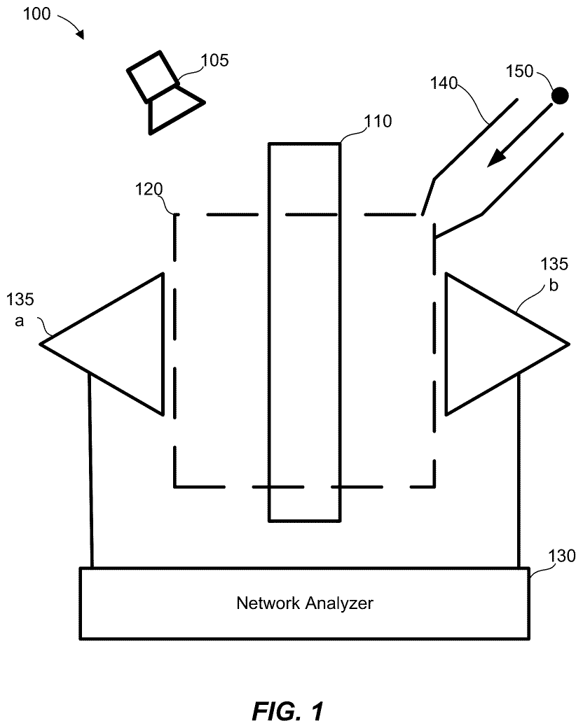

In one embodiment, as illustrated in FIG. 1, an airborne particle sensor 100 for detecting a target particle 150 can comprise: a field generator source 105, a resonator 120, a receiver 130 (e.g., a network analyzer), or a combination thereof. The field generator source can be configured to emit an incident electromagnetic field. The resonator 120 can have a focusing structure configured to focus the incident electromagnetic field in a gap region (e.g., in an area within 110) that is configured to accept the target particle 150. The receiver 130 can be configured to detect a resonant signal from the resonator 120. The resonant signal can shift due to presence of the target particle 150 in the gap region.

The airborne particle sensor 100 in FIG. 1 can further comprise a particle filter 110 that can capture a target particle 150 within a resonator 120 or resonator cavity. The network analyzer 130 can comprise a first port 135a, a second port 135b. An inlet 140 can be configured to facilitate entry of a target particle 150.

The resonator 120 can comprise any suitable resonator. In one example, the resonator 120 can include at least one of: a Fabry-Perot (F-P) resonator, a coaxial resonator, a dielectric resonator, a crystal resonator, a ceramic resonator, a surface acoustic wave (SAW) resonator, and an yttrium iron garnet (YIG) resonator, the like, or a combination thereof.

The gap region can be configured to capture a target particle 150 and shift a resonant signal due to the capturing of the target particle 150. In one example, the gap region can be a capacitive gap region having a capacitive gap of from 0.1 μm to about 1 mm. In another example, the capacitive gap can be from 1 μm to about 100 μm. In one more example, the capacitive gap can be from 1 μm to about 25 μm. In some examples, the capacitive gap can be 1 μm, 2 μm, 4 μm, 8 μm, 12 μm, 15 μm, 20 μm, or the like.

The gap region can also be an inductive gap region. In one example, the inductive gap region can have a range of from 0.1 μm to about 1 mm. In another example, the inductive gap region can be from 1 μm to about 100 μm. In one more example, the inductive gap region can be from 1 μm to about 25 μm. In some examples, the inductive gap region can be 1 μm, 2 μm, 4 μm, 8 μm, 12 μm, 15 μm, 20 μm, or the like.

The wavelength of the incident electromagnetic field can be selected to be greater than a length, width, depth, area, volume, or the like of the sample (having the target particle 150) captured by the resonator 120. In one example, the wavelength of the incident electromagnetic field can be greater than 30× of the sample at 10 GHZ, and in some cases greater than 100 s x of the sample at 10 GHZ.

The network analyzer 130 can be configured to detect the changes in resonance upon capture of the target particle 150. In one aspect, the network analyzer 130 can be operatively connected to the receiver to record responses. In one example, the network analyzer 130 can be configured to calculate one or more of: a transmission coefficient of the resonant signal before or when a target particle 150 is accepted within the gap region: a resonator frequency shift before or when a target particle 150 is accepted within the gap region: an amplitude modulation of the resonant signal before or when a target particle 150 is accepted within the gap region: or a phase shift of the resonant signal before or when a target particle 150 is accepted within the gap region: the like, or a combination thereof.

The sensitivity of the airborne particle sensor 100 can be adequate to detect a change in resonance upon capture of a target particle 150. In one example, the airborne particle sensor 100 can have a sensitivity of one or more of: from about 10 MHz/1 μL of pathogen sample to about 1000 MHz/1 μL of pathogen sample. In another example, the airborne particle sensor can have a sensitivity of one or more of: from about 25 MHZ/1 μL of pathogen sample to about 250 MHz/1 μL of pathogen sample. In another example, the airborne particle sensor can have a sensitivity of one or more of one or more of: 30 MHz/1 μL of pathogen sample: 40 MHz/1 μL of pathogen sample: 50 MHz/1 μL of pathogen sample: 60 MHz/1 μL of pathogen sample: 70 MHz/1 μL of pathogen sample: 80 MHz/1 μL of pathogen sample: 90 MHz/1 μL of pathogen sample: 100 MHz/1 μL of pathogen sample: 110 MHz/1 μL of pathogen sample: 120 MHz/1 μL of pathogen sample: 130 MHZ/1 μL of pathogen sample: 140 MHz/1 μL of pathogen sample: 150 MHz/1 μL of pathogen sample: 200 MHz/1 μL of pathogen sample: the like, or combinations thereof.

In another example, the airborne particle sensor 100 can have a sensitivity of from about 100 kHz/mm³ of dielectric sample to about 50 MHz/mm³ of dielectric sample. In another example, the airborne particle sensor can have a sensitivity of from about 500 KHz/mm³ of dielectric sample to about 10 MHz/mm³ of dielectric sample. In another example, the airborne particle sensor can have a sensitivity of one or more of: about 750 KHz/mm³ of dielectric sample: about 1 MHz/mm³ of dielectric sample: about 2 MHZ/mm³ of dielectric sample: about 3 MHz/mm³ of dielectric sample: about 4 MHZ/mm³ of dielectric sample: about 5 MHz/mm³ of dielectric sample: about 8 MHz/mm³ of dielectric sample: about 10 MHz/mm³ of dielectric sample: the like: or a combination thereof.

In yet another example, the airborne particle sensor 100 can have a sensitivity of from about 1 MHz/mm³ of metal sample (e.g., soot) to about 100 MHz/mm³ of metal sample. In one example, the airborne particle sensor can have a sensitivity of from about 5 MHz/mm³ of metal sample to about 50 MHz/mm³ of metal sample. In another example, the airborne particle sensor can have a sensitivity of one or more of: 10 MHZ/mm³ of metal sample: 15 MHz/mm³ of metal sample: 20 MHz/mm³ of metal sample: 25 MHz/mm³ of metal sample: 30 MHz/mm³ of metal sample; the like: or a combination thereof.

In another aspect, the airborne particle sensor 100 can comprise any suitable particle filter 110 for capturing target particles 150 within a gap region. A paper filter material 110, permeable mesh structure 110, or other particle capture medium can be located at the center of the resonator 120 where the standing wave electric field is maximum, although a functional device may vary from optimal (e.g., such as within 5%, 10% or within 25% of maximum depending on design criteria and chosen materials). In one aspect, the particle filter 110 can be a porous layer positioned within 25% of a maximum of a standing wave of the incident electromagnetic field. In another aspect, the particle filter 110 can be a porous layer positioned within 10% of a maximum of a standing wave of the incident electromagnetic field. In another example, the particle filter 110 can be a porous layer positioned within 5% of a maximum of a standing wave of the incident electromagnetic field. In another example, the particle filter 110 can be a porous layer positioned within 1% of a maximum of a standing wave of the incident electromagnetic field.

The airborne particle sensor 100 can have any suitable particle filter 110 that can exhibit a change in resonance upon capture of a target particle 150. The mesh/paper filter 110 can be an atomic layer of graphene or other suitable 2-D layered materials with specific sensitivity to the particles of interest. The conductivity of the atomic layer materials can change when charged particles deposit on them, increasing the resonator's sensitivity. In one example, the particle filter 110 can be at least one of an atomic 2D layer, paper, mesh, the like, or a combination thereof. In another example, the particle filter 110 can be functionalized with an aptamer which selectively binds with a target particle 150.

The porous layer 110 can have suitable dimensions for orientation within a resonant cavity while facilitating capture of a target particle 150. In one example, the porous layer 110 can have a height, width, depth, or a combination thereof ranging from about 0.1 cm to about 25 cm. In another example, the porous layer 110 can have a height, width, depth, or a combination thereof ranging from about 0.5 cm to about 10 cm. In one more example, the porous layer 110 can have a height, width, depth, or a combination thereof ranging from about 1 cm to about 5 cm. In another example, the porous layer 110 can have a height, width, depth, or a combination thereof that can be 1.5 cm, 2.0 cm, 2.5 cm, 3.0 cm, 3.5 cm, 4.0 cm, 4.5 cm, 5 cm, the like, or a combination thereof.

The target particle 150 can be any particle that can exhibit a change in resonance when captured by the airborne particle detector 100. In one example, the target particle 150 can comprise a pathogen that is a virus, a bacterium, a fungus, the like, or a combination thereof. In another example, the target particle can comprise a dielectric sample. In one more example, the target particle can be a metal (e.g., iron). Further, dielectrics such as silica (sand) particles or soot (carbon particles from car emission), and the like can also be used. Different particles are differentiated by their signature electromagnetic properties and physical size.

Metamaterials (MTMs) are composite structures engineered to exhibit electromagnetic properties like negative permittivity and permeability, and negative or zero refractive indices. MTMs can be used for sensor applications. In these sensors, the measurand (sensed sample) can changes the sensor's resonance and or the amplitude of its reflection/transmission coefficients. For sensors with coupled resonant modes, the measurand can change the strength of the coupling, which can lead to frequency splitting. and/or amplitude modulation of a harmonic signal.

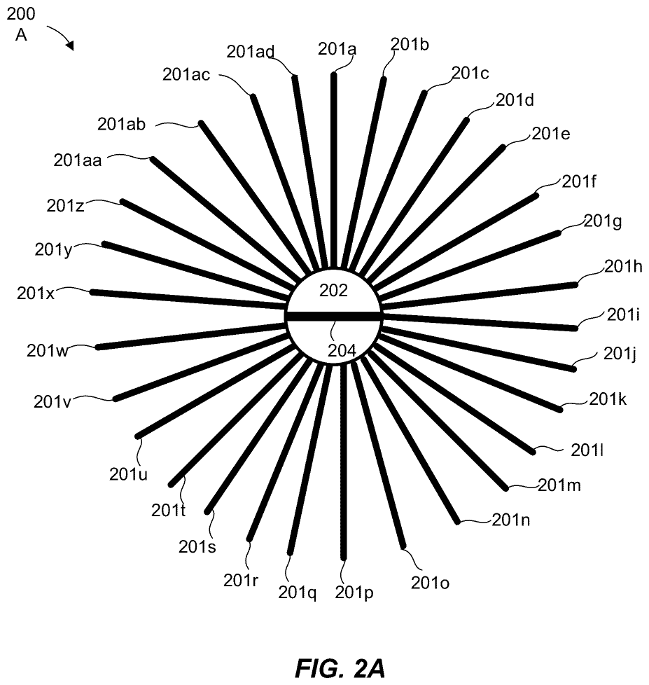

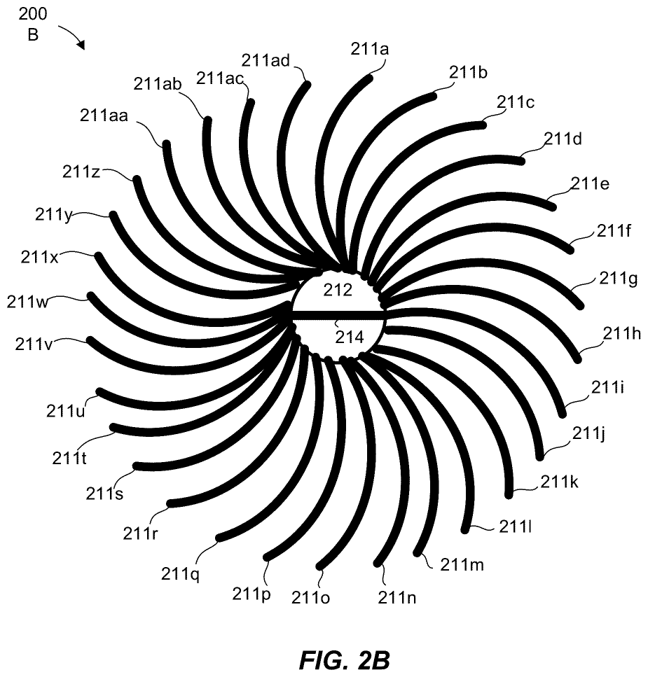

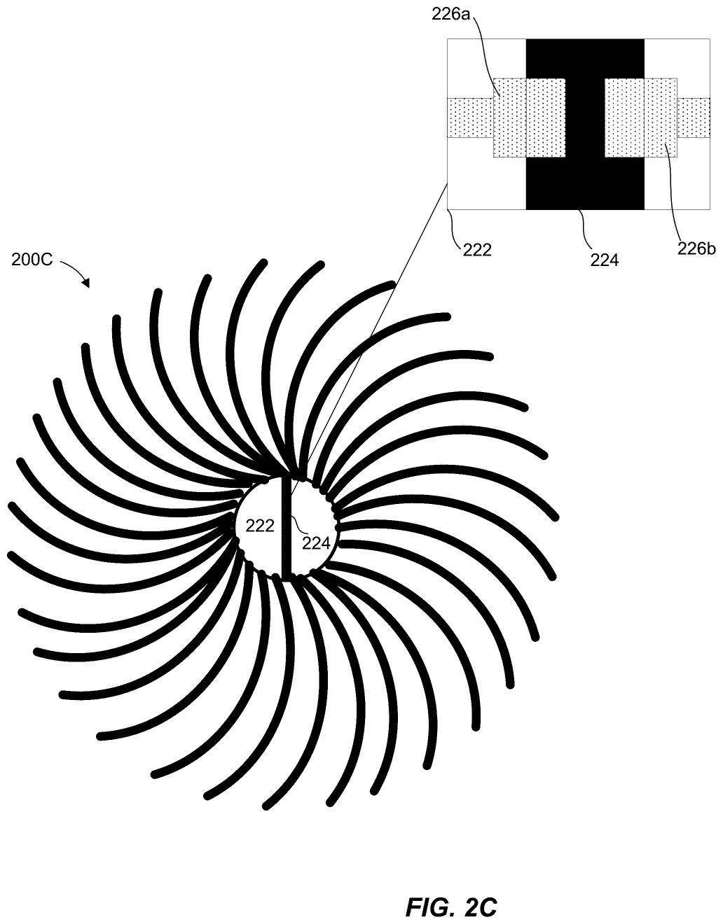

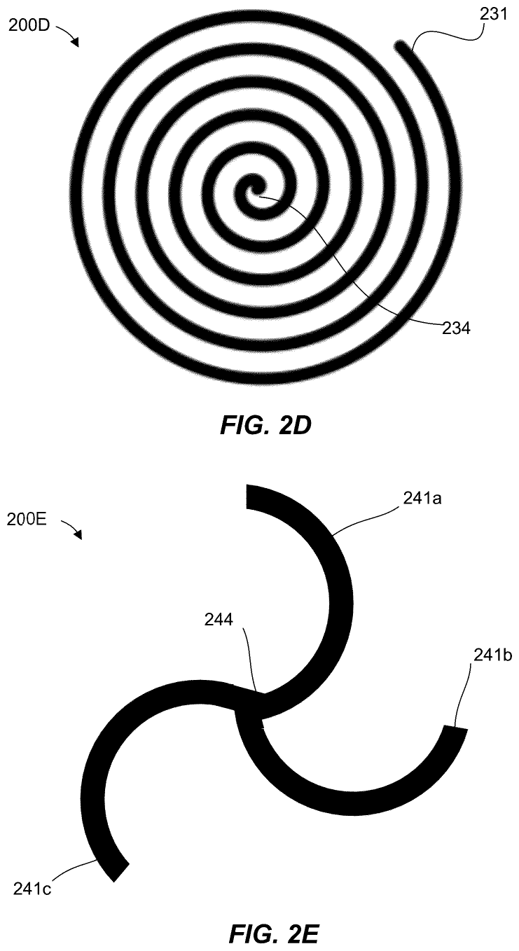

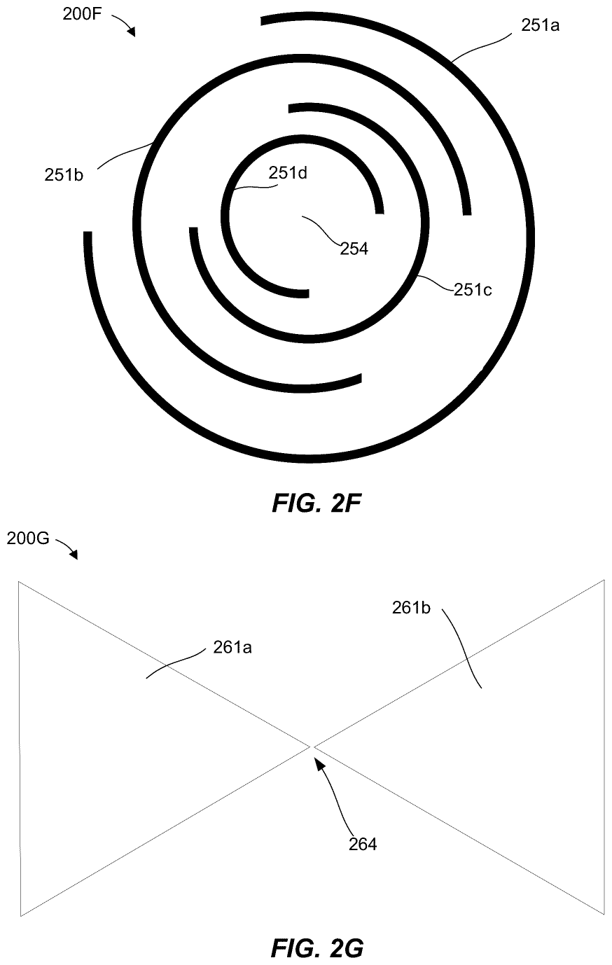

In one example, the MTM structure can be an X-band device with radial spokes connected to a central split hub with a gap region. This general principle is illustrated in FIGS. 2A to 21. Spokes at different angles can allow the structure to interact with incident electromagnetic waves with different polarizations and different directionality. A periodic array can provide MTM surfaces with distributed gap regions for distributed sensing of viral and other particles. The spokes can form electric dipoles with different polarization angles that generate large electric fields in the gap region when illuminated with an electromagnetic wave. Large fields in the gap can be a focused spot for detecting airborne particles. When compared to terahertz waves with large attenuation in humid air, x-band signals can have small attenuation and can be used to remotely interrogate surface sensors from long distances (e.g., greater than 100 m) while providing significant cost savings.

In one embodiment, the airborne particle sensor 100 can comprise a resonator 120 that can comprise one or more X-band metamaterials having a selected conductivity, a selected capacitance, the like, or a combination thereof. In one example, a difference in the conductivity can be measured, identified and reported before and after exposure. In another example, a difference in the capacitance can be measured, identified and reported.

In one example, as illustrated in FIGS. 2A to 2C, the focusing structure 200A, 200B, 200C can comprise a plurality of radial spokes (e.g., 201a to 201ad, 211a to 211ad) connected to a central hub (e.g., 202, 212, 222) housing the gap region (204, 214, 224). In one aspect, the one or more radial spokes can be flat (e.g., 201a to 201ad) or curved (e.g., 211a to 211ad). In another aspect, the central hub (e.g., 202, 212, 222) can be split (202, 212, 222). In another aspect, the central hub can be solid (e.g., without the gap region 204, 214, 224).

As illustrated in FIG. 2A, the focusing structure 200A of the resonator can comprise: a central hub 202 housing the gap region 204, and a plurality of radial spokes 201a to 201ad. The resonator can be an electric dipole structure with one or more dipoles with varying lengths produced by the angle each spoke makes with an incident electromagnetic field (e.g., a vertically polarized electric field). In this example, there are 30 spokes 201a to 201ad centrally connected to a split hub 202 with a gap 204.

In one example, the focusing structure can comprise a central hub that is split. In one example, as illustrated in FIG. 2A, a split circular disc 204 (e.g., 1.1 mm diameter) at the central hub 202 can have a capacitive gap region (e.g., 0.004 mm) between the upper and lower halves of the central hub 202 of the structure.

In another example, the focusing structure 200B, 200C of the resonator can comprise: a central hub 212, 224 housing the gap region 214, 224, and a plurality of curved radial spokes 211a to 211ad. In this example, there are 30 spokes 211a to 211ad centrally connected to a split hub 212, 222 with a gap 214, 224. In one example, the split has a horizontal orientation (e.g., 214). In another example, the split has a vertical orientation (e.g., 224). As illustrated in FIG. 2C, the split hub 222 can comprise a vertically oriented gap region 224, a first port 226a and a second port 226b.

In another example, as illustrated in FIGS. 2D to 2F, the focusing structure 200D, 200E, 200F can comprise a spiral structure comprising a central space 234, 244, 254 housing the gap region. In one example, the spiral structure 200D, 200E can be continuous. In another example, the spiral structure 200F can be discrete. When the spiral structure 200D is continuous, the structure 200D can comprise a spiraling member 231 that terminates at the central space 234. In another example, the spiral structure 200E can be continuous with a plurality of arms 241a, 241b, 241c joined at the central space 244. In another example, when the spiral structure 200F is discrete, the plurality of arms 251a, 251b, 251c, 251d can be discrete arms that partially or fully surround a central space 254.

In another example, as illustrated in FIG. 2G, the focusing structure 200G can comprise an hourglass structure comprising a central point 264 housing or defining the gap region. In one example, the hourglass structure can comprise a first triangular structure 261a and a second triangular structure 261b that can directly or indirectly couple to the second triangular structure at the central point 264.

In another example, as illustrated in FIG. 2H, the focusing structure 200H can comprise a distributed planar structure comprising a plurality of distributed gap regions (e.g., 274a, 274b, 274c) and a plurality of arms (e.g., 271a, 271b, 271c, 271d). As illustrated in FIG. 2I, the gap regions (e.g., 284a) of the flat structure 200I can exhibit a particular resonance that can be detected relative to the resonance exhibited when the target particle is not present.

In one example, the dimensions of the focusing structure can be any suitable dimensions that, when combined with the gap region, generate an electric dipole suitable for sensing the target particle (e.g., dielectric samples or resistive samples such as pathogens, soot, dust, or the like). In one example, the height, width, or a combination thereof of the focusing structure can range from about 1 mm to about 100 mm. In another example, the height, width, or a combination thereof of the focusing structure can range from about 5 mm to about 50 mm. In one more example, the height, width, or a combination thereof of the focusing structure can range from about 10 mm to about 20 mm. In one example, the height, width, or a combination thereof of the focusing structure can be about 12 mm, about 14 mm, about 16 mm, about 18 mm, the like, or a combination thereof. In a specific example, the height and width of the focusing structure can be 16 mm×16 mm.

In another example, the thickness of the focusing structure can range from about 0.1 mm to about 10 mm. In one example, the thickness of the focusing structure can range from about 0.5 mm to about 5 mm. In another example, the thickness of the focusing structure can range from about 0.75 mm to about 1.25 mm. In one specific example, the thickness of the focusing structure can be about 1 mm.

Although two-dimensional structured are exemplified, three-dimensional focusing structures can also be used as long as there is a diffusion pathway for contact with portions of the focusing structure. As non-limiting examples, a hemispherical spoked structure, asymmetric shaped structure, or other 3D structure can be used as the focusing structure. It is also noted that the target particle can vary the measured response when located in the gap. In some cases, the target particle can also vary the measured response when located elsewhere on the focusing structure, i.e. not at the gap.

In one example, the radial spokes or the arms can have a length, width, diameter, or a combination thereof suitable for generating a half wavelength dipole at a selected incident electromagnetic field. In one example, the length of the radial spokes or arms can be in a range from about 0.5 mm to about 100 mm. In another example, the length of the radial spokes or arms can be in a range from about 5 mm to about 25 mm. In another example, the length of the radial spokes or arms can be one or more of about 6 mm, about 8 mm, about 10 mm, about 12 mm, about 14 mm, about 16 mm, about 18 mm, about 20 mm, about 22 mm, about 24 mm, the like, or a combination thereof.

In one example, the width or diameter of the radial spokes or arms can be in a range from about 0.1 mm to about 20 mm. In another example, the width or diameter of the radial spokes or arms can be in a range from about 0.5 mm to about 10 mm. In one more example, the width or diameter of the radial spokes or arms can be in a range from about 1 mm to about 5 mm. In one specific example, the width or diameter of the radial spokes or arms can be 2 mm, which can generate a half wavelength dipole at 10 GHz.

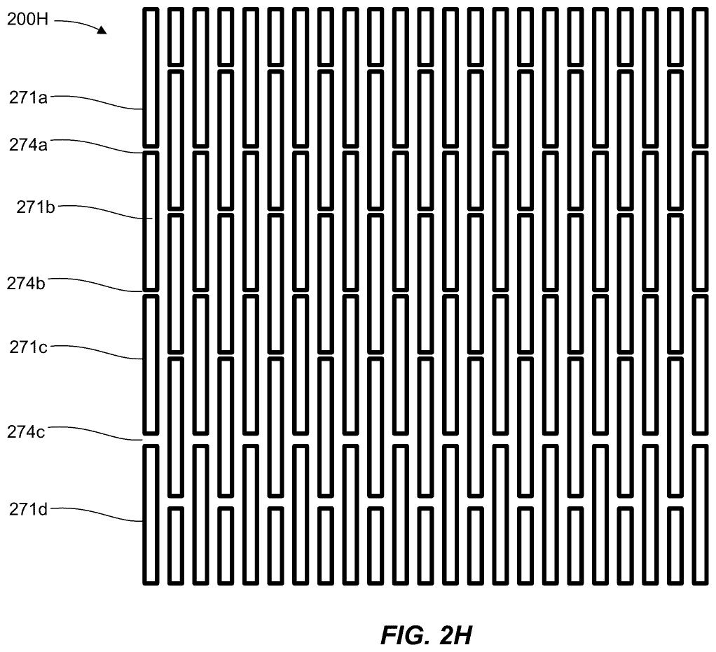

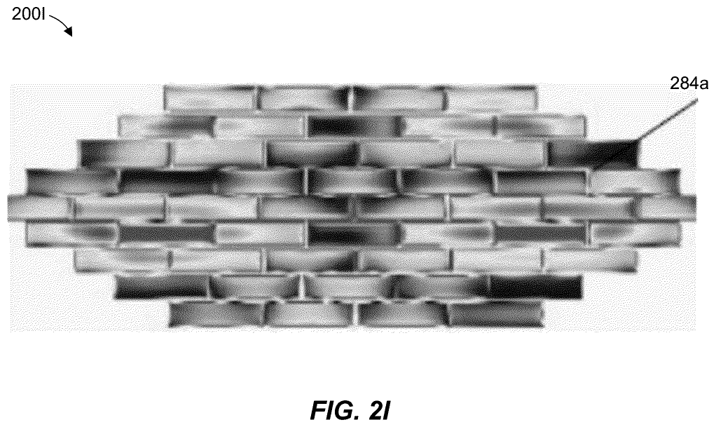

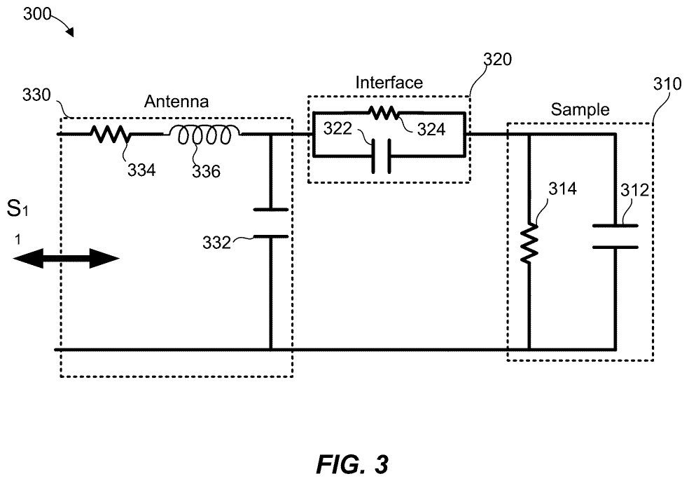

The spoke structure, as illustrated in FIG. 2A, can be viewed as an electric dipole antenna. Near one of its resonances a simple lumped circuit model shown in FIG. 3 can be used to study its interactions with particles in the gap region. A non-magnetic sample in the gap region is modeled as an RC circuit 310 having a resistance of Rₛ 314 and a capacitance of Cₛ 312 as calculated as the frequency of operation. The interface 320 between the sample 310 and the MTM structure 330 can have a resistance Rᵢ 324 and capacitance Cᵢ 322 and can electrically model the MTM-sample interaction. The MTM structure 330 can be modeled as having the capacitance 332, the inductance 336, and the resistance 334. The transmission coefficient, S11, can be calculated from this equivalent circuit diagram 300.

The resonant MTM sensor can be a complex structure (as illustrated in FIGS. 2A to 21) with multiple resonances. Near a frequency of operation of about 10 GHz, the electrical behavior can be approximated as a simple 2^{nd} order resonant system with resonant frequency

$${f}_{0} = 1 / 2 π \sqrt{( {L}_{r} {C}_{r} )} ,$$

and with an impedance that is

$${Z}_{r} = {R}_{r} + j ω {L}_{r} + \frac{1}{j ω {C}_{r}} .$$

Assuming the impedance of the non-magnetic sample in FIG. 3 is

$$\frac{1}{{Z}_{S}} = \frac{1}{{R}_{S}} + \frac{1}{j ω C s} ,$$

then, the Rₛ and Cₛ of 1 μL of water (approximately 1 mm³ volume) can be estimated by modeling the water droplet as a sphere with a radius of ‘r.’ Consequently, the radius of the droplet can be modeled as (4/3)*pi*r³=1 mm³, which can be used to calculate r-0.62 mm. The area covered by the droplet can also be estimated as A=4*pi*r²=4.83 mm².

### Since

$$C = {ε}_{0} {ϵ}_{r} * ( area / distance )$$

and the average distance in the gap can be about d=0.85 mm (which can be larger than 0.004 mm of the split disk gap due to horizontal spokes), the capacitances for water and saliva can be calculated as

$${C}_{w a t e r} = 3.92 pF and$$

$${C}_{saliva} = 3.82 pF , or$$

$$\sqrt{\frac{{C}_{w a t e r}}{{C}_{{C}_{saliva}}}} = 1.01 ,$$

when the water sample has a relative permittivity of ε_{water}˜78 and the saliva has a relative permittivity of εₛₐₗᵢᵥₐ˜76.

The ƒ₀ with water (ƒ_{water}) or saliva (ƒₛₐₗₙₐ) can be indirectly proportional to the square root of the sensor's capacitance at resonance. When measured, the experimental frequencies for 11 μL water and saliva were respectively 10.1762 GHz and 10.17172 GHz.

Thus,

$$\frac{{f}_{saliva}}{{f}_{water}} = 1.004 .$$

When the relative permittivity of εₛₐₗᵢᵥₐ˜76 rather than ˜76.6, then

$$\frac{{F}_{saliva}}{{F}_{water}} = \sqrt{\frac{{C}_{water}}{{C}_{{C}_{saliva}}}} = 1.004 .$$

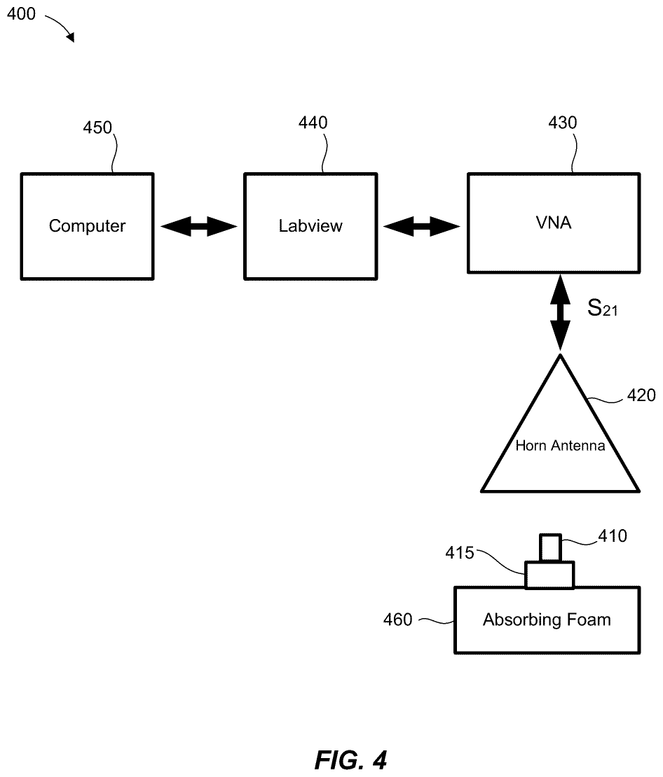

In one example, as illustrated in FIG. 4, an experimental setup 400 can be used. A vector network analyzer (VNA) 430 (e.g., an HP-8720C VNA) and a horn antenna 420 (e.g., an X-band horn antenna) can be used to measure the S21 of the sensor 415 (e.g., an MTM sensor) situated directly under the horn antenna 420. An absorbing foam 460 under the sensor 415 can reduce reflection from the benchtop. A data acquisition system comprising the VNA 430, LabVIEW 440, and a computer 450 can collect the measured data from the sample 410 (e.g., a 1 μL deionized water sample).

Some electronic sensors can spatially sample air within their immediate vicinity but covering large volumes uses fans and funnels that add to the sensor noise and cost.

Therefore, in one example, measurements with microwaves operating in a range of about 5 GHz to about 20 GHz can cover relatively large volumes (proportional to the wavelength cubed, λ³, wherein 2<λ<3.75 cm) while also having high sensitivity.

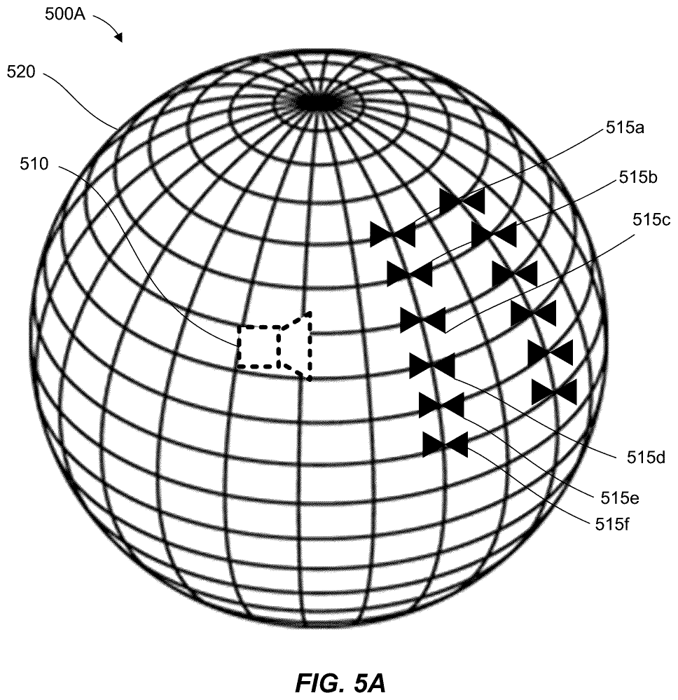

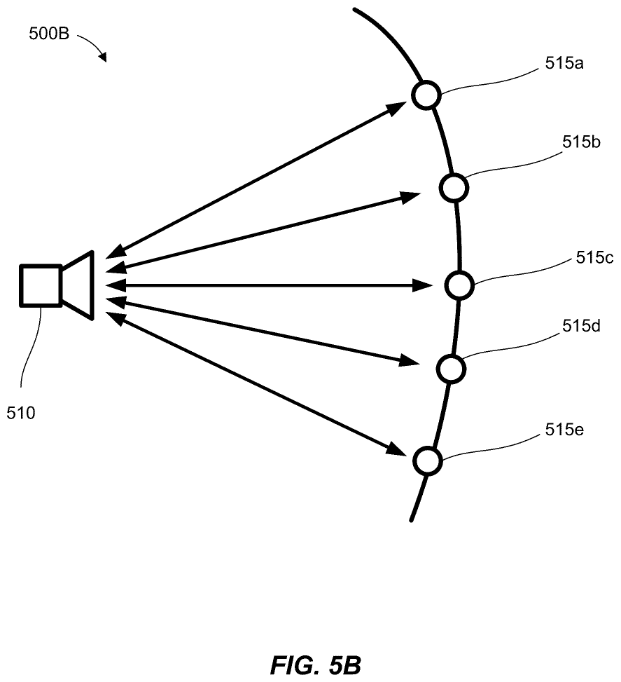

In one embodiment, an airborne particle sensing system 500A, 500B can comprise a source 510, an array of airborne particle sensors 515a, 515b, 515c, 515d, 515e, 515f, and a receiver (e.g., 510 when the source and receiver are co-located), as illustrated in FIGS. 5A and 5B. In one aspect, the source can be configured to emit an incident electromagnetic field having a frequency as otherwise disclosed herein. In another aspect, the array of airborne particle sensors 515a, 515b, 515c, 515d, 515e, 515f can be housed in a ventilated structure 520, wherein each airborne particle sensor 515a, 515b, 515c, 515d, 515e, 515f can comprise a resonator having a focusing structure configured to focus the incident electromagnetic field in a gap region that is configured to accept a target particle, as disclosed herein. In yet another aspect, the receiver can be configured to detect the resonant signals from the array of airborne particle sensors 515a, 515b, 515c, 515d, 515e, 515f. The receiver can be integrated with or can be separate from the source 510.

In one example, the sensors 515a, 515b, 515c, 515d, 515e, 515f can include microwave sensor element arrays, including an array of microwave virus sensors. Although other sensor configurations may be suitable, sensors can include a gap region within which particles can be trapped or bind with suitable functional groups such as aptamers. In one example, the sensor elements can include x-band metamaterial sensors which can be arranged within a microwave radiation pathway as shown generally in FIGS. 2A to 21. One or more electrodes can be connected to each of the sensor elements to measure a a conductivity and/or capacitance response from each of the sensor elements.

In one example, the airborne particle sensor can comprise a source configured to emit an incident electromagnetic field having a frequency capable of traversing a selected distance of up to about 500 m without substantial attenuation. In another example, the frequency can be capable of traversing a selected distance of up to about 100 m without substantial attenuation. In another example, the frequency can be capable of traversing a selected distance of up to one or more of about 5 m, 10 m, 20 m, 30 m, 40 m, 50 m, or a combination thereof without substantial attenuation. In one example, the amount of attenuation that is substantial can be an attenuation amount of greater than 5%, 10%, 20%, 30%, 40%, 50%, 60%, 70%, 80%, the like, or a combination thereof when calculated as a percentage decrease compared to the incident electromagnetic field. The emitted electromagnetic wave is transmitted from the source and reflected to the source that uses an appropriate circuit (a circulator or directional couplers) to separate the outgoing transmitted wave from the reflected incoming wave similar to radar. The change experienced by the received wave is due both the particles captured by the sensing structure and by the changes that may occur in the intervening space between the transceiver and the sensing surface. To separate these two changes, a reflecting surface oriented next to the sensing surface can be used to generate a reference beam that is largely or exclusively only affected by the intervening space. The combination of the reference beam and the reflected beam (electromagnetic wave reflected from the sensing surface) can be used to cancel out the effect of the intervening space and produce a signal that is largely or exclusively only affected by the particles captured by the sensing surface. Signal-to-noise ratios as small as 0.1 can be easily tolerated by this differential detection method.

In one example, the source can be configured to emit the incident electromagnetic field having a microwave frequency ranging from about 5 GHz to about 20 GHZ. In another example, the microwave frequency can have a range of from about 5 GHz to about 15 GHZ. In one more example, the incident electromagnetic field can be an X-band electromagnetic field (i.e., a frequency ranging from about 8 GHz to about 12 GHZ).

In another example, the source can be configured to emit an incident electromagnetic field that traverses a smaller volume when compared to the volume traversed by a microwave frequency. In this example, the frequency can be a terahertz frequency ranging from about 0.75 GHz to about 0.83 GHZ.

In another example, the array of airborne particle sensors 515a, 515b, 515c, 515d, 515e, 515f can be configured to sample a selected volume of air in a selected time. In one aspect, the time can be selected based on a threshold detectability, selectivity, sensitivity, confidence level, or a combination thereof for a target particle. In one aspect, the selected time can be less than 10 minutes, 5 minutes, 2 minutes, 1 minute, 30 seconds, 15 seconds, 5 seconds, the like, or a combination thereof.

In one more example, the ventilated structure 520 can be any structure that is suitable for receiving a threshold amount of target particles within a threshold volume within a threshold amount of time. In one example, the ventilated structure 520 can be one or more of planar, three-dimensional, curved, flat, or a combination thereof. The height, width, depth, diameter, the like, or a combination thereof of the ventilated structure can have a range of: from about 0.1 mm to about 10 m: from about 1 mm to about 1 m: 1 cm to about 10 cm; the like: or a combination thereof. The ventilated structure is a guide for the eye and the sensors are mounted on a (for example) a stadium wall over a segment of a spherical surface so that a central transceiver can interrogate different sensors easily.

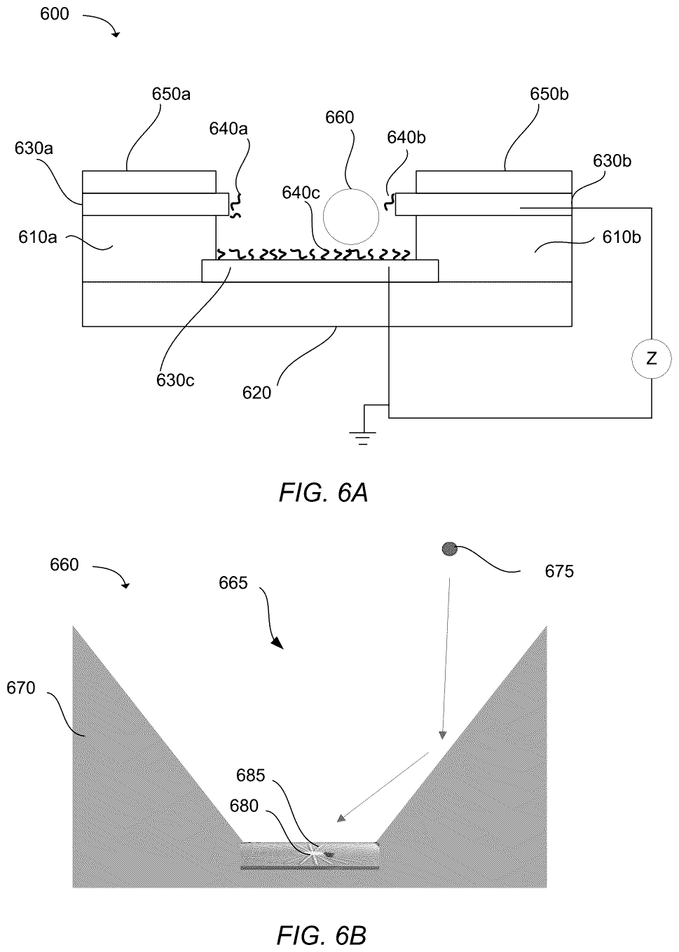

In another example, as illustrated in FIG. 6A, the gap region, mesh/paper filter, or MTMs can be further functionalized with appropriate molecules or aptamers to bind with specific airborne particles. A sensor structure 600 can comprise: a substrate 620 (e.g., Si), a first electrode 610a (e.g., SiO₂), a second electrode 610b (e.g., SiO₂), a layer 610a, 610b of an electrically conductive solid (e.g., gold), a layer of aptamers 640a, 640b, and 640c a hydrophobic material 650a and 650b (e.g., a layer of photoresist), the like, or combinations thereof.

In one example, the sensor structure 600 having an opening into which a target particle can enter. Aptamers 640a, 640b, 640c can be configured to bind to receptors on a specifically selected target particle 660. Upon binding of the target particle 660 to the aptamers 640a, 640b, 640c, a circuit Z is completed between adjacent electrodes 630b, 630c and a signal can be generated. The sensor structure 600 can further comprise an additional electrode 630a to complete a circuit when the target particle 660 binds to a different portion of the structure. Further examples of such sensor structures are described in U.S. Pat. No. 11,009,482 which is incorporated herein by reference.

In another example, as illustrated in FIG. 6B, the airborne particle sensor 660 can further comprise an inlet 665 having a funnel structure 670 configured to direct a sample 675 having the target particle into the gap region 680 of the focusing structure 685 while also increasing concentration of particles within collected air samples. This particle sensor 660 can be operated alone or as part of an array of particle sensors 660 to form a network of dozens, hundreds, or thousands of sensors. In some case, each sensor can have a corresponding funnel structure. Alternatively, a subset of sensors, or the entire array can have a single funnel structure. In yet another alternative, at least a portion of an inner surface of the funnel structure can have electromagnetically reflective material (e.g. metallic) to allow further focusing of electromagnetic signals.

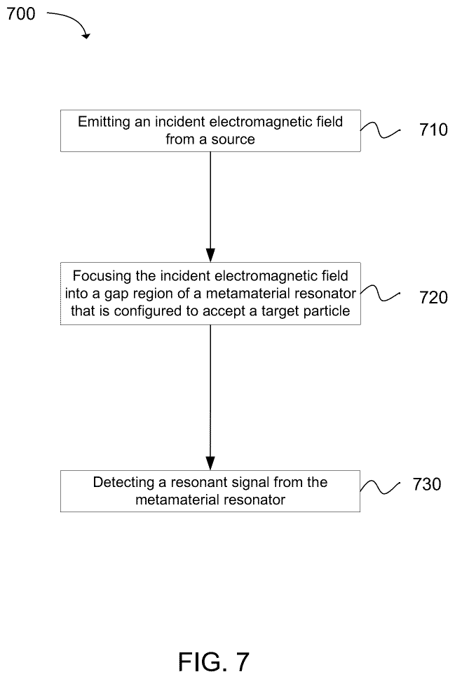

FIG. 7 illustrates a flow diagram of a method according to the present technology. For simplicity of explanation, the method is depicted and described as a series of acts. However, acts in accordance with this disclosure can occur in various orders and/or concurrently, and with other acts not presented and described herein. Furthermore, not all illustrated acts may be used to implement the methods in accordance with the disclosed subject matter.

In one embodiment, as illustrated in FIG. 7, a method 700 for sensing airborne particles can comprise emitting an incident electromagnetic field from a source, as shown in block 710. In one aspect, the method can comprise focusing the incident electromagnetic field into a gap region of a resonator that is configured to accept a target particle, as shown in block 720. In another aspect, the method can comprise detecting a resonant signal from the resonator, as shown in block 730.

In one example, the method can comprise one calculating a resonator frequency shift when the target particle is accepted within the gap region. In another example, the method can comprise correlating the resonator frequency shift with a capture of the target particle in the gap region. In another example, the method can comprise calculating a transmission coefficient of the gap region. In another example, the method can comprise calculating an amplitude modulation of the resonant signal. In another example, the method can comprise calculating a phase shift of the resonant signal.

In another example, the method can comprise focusing the incident electromagnetic field into the gap region using a focusing structure comprising a metamaterial. In one example, the metamaterial can be selected from one or more of: a plurality of radial spokes connected to a central hub housing the gap region, wherein each radial spoke is flat or curved, and wherein the central hub is split or solid: or a spiral structure comprising a central space housing the gap region, wherein the spiral structure is continuous or discrete: or an hourglass structure comprising a central point housing the gap region: or a flat structure comprising a plurality of distributed gap regions: the like: or a combination thereof.

In another example, the wavelength of the incident electromagnetic field can be greater than a length of a sample captured by the resonator in which the sample includes 0) the target particle. In one example, the method can comprise emitting: a microwave frequency ranging from about 5 GHz to about 20 GHz: a terahertz frequency ranging from about 0.75 GHz to about 0.83 GHZ: a microwave frequency that is an X-band electromagnetic field: or a frequency capable of traversing a selected volume with a threshold sensitivity.

In another example, the method can comprise directing a sample having the target particle into the gap region. In another aspect, the method can comprise positioning a particle filter at a standing wave electric field within the gap region. In another aspect, the method can comprise accepting a target particle at particle filter oriented within the gap region. In another aspect, the method can comprise sending a response to a network analyzer operatively connected to the resonator.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Various techniques, or certain aspects or portions thereof, can take the form of program code (i.e., instructions) embodied in tangible media, such as floppy diskettes, compact disc-read-only memory (CD-ROMs), hard drives, non-transitory computer readable storage medium, or any other machine-readable storage medium wherein, when the program code is loaded into and executed by a machine, such as a computer, the machine becomes an apparatus for practicing the various techniques. Circuitry can include hardware, firmware, program code, executable code, computer instructions, and/or software. A non-transitory computer readable storage medium can be a computer readable storage medium that does not include signal. In the case of program code execution on programmable computers, the computing device can include a processor, a storage medium readable by the processor (including volatile and non-volatile memory and/or storage elements), at least one input device, and at least one output device. The volatile and non-volatile memory and/or storage elements can be a random-access memory (RAM), erasable programmable read only memory (EPROM), flash drive, optical drive, magnetic hard drive, solid state drive, or other medium for storing electronic data. The low energy fixed location node, wireless device, and location server can also include a transceiver module (i.e., transceiver), a counter module (i.e., counter), a processing module (i.e., processor), and/or a clock module (i.e., clock) or timer module (i.e., timer). One or more programs that can implement or utilize the various techniques described herein can use an application programming interface (API), reusable controls, and the like. Such programs can be implemented in a high-level procedural or object-oriented programming language to communicate with a computer system. However, the program(s) can be implemented in assembly or machine language, if desired. In any case, the language can be a compiled or interpreted language, and combined with hardware implementations.

As used herein, the term processor can include general purpose processors, specialized processors such as VLSI, FPGAs, or other types of specialized processors, as well as base band processors used in transceivers to send, receive, and process wireless communications.

It should be understood that many of the functional units described in this specification have been labeled as modules, in order to more particularly emphasize their implementation independence. For example, a module can be implemented as a hardware circuit comprising custom very-large-scale integration (VLSI) circuits or gate arrays, off-the-shelf semiconductors such as logic chips, transistors, or other discrete components. A module can also be implemented in programmable hardware devices such as field programmable gate arrays, programmable array logic, programmable logic devices or the like.

In one example, multiple hardware circuits or multiple processors can be used to implement the functional units described in this specification. For example, a first hardware circuit or a first processor can be used to perform processing operations and a second hardware circuit or a second processor (e.g., a transceiver or a baseband processor) can be used to communicate with other entities. The first hardware circuit and the second hardware circuit can be incorporated into a single hardware circuit, or alternatively, the first hardware circuit and the second hardware circuit can be separate hardware circuits.

Modules can also be implemented in software for execution by various types of processors. An identified module of executable code can, for instance, comprise one or more physical or logical blocks of computer instructions, which can, for instance, be organized as an object, procedure, or function. Nevertheless, the executables of an identified module need not be physically located together but can comprise disparate instructions stored in different locations which, when joined logically together, comprise the module and achieve the stated purpose for the module.

Indeed, a module of executable code can be a single instruction, or many instructions, and can even be distributed over several different code segments, among different programs, and across several memory devices. Similarly, operational data can be identified and illustrated herein within modules and can be embodied in any suitable form and organized within any suitable type of data structure. The operational data can be collected as a single data set or can be distributed over different locations including over different storage devices, and can exist, at least partially, merely as electronic signals on a system or network. The modules can be passive or active, including agents operable to perform desired functions.

The following examples are provided to promote a clearer understanding of certain embodiments of the present disclosure and are in no way meant as a limitation thereon.

### Example 1: Viral Infected and Uninfected Saliva on Plain Graphene and Filter Paper

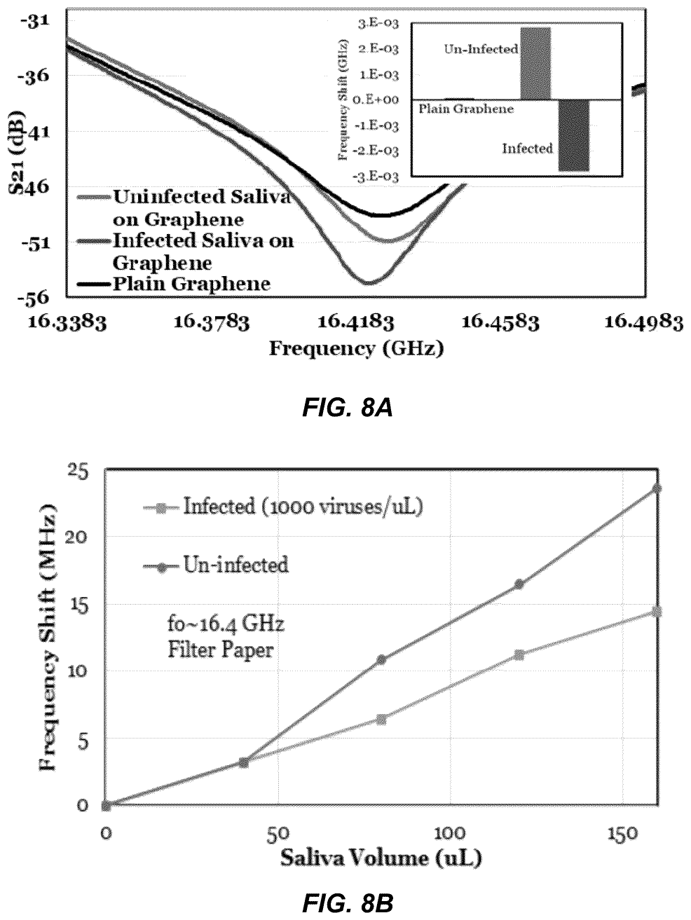

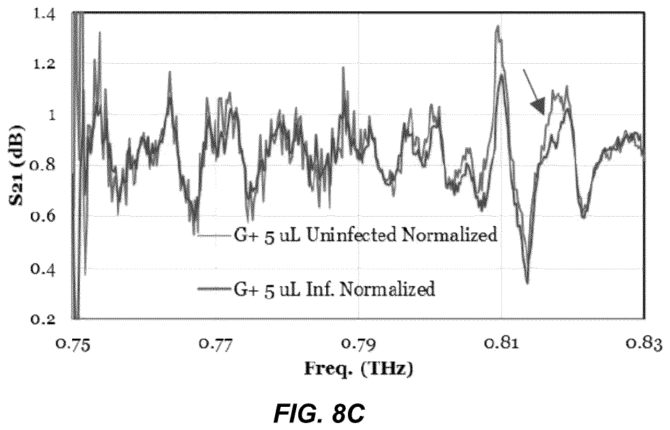

FIG. 8A shows the transmission coefficient, S21 (dB), of a plain CVD graphene with 5 μL of COVID-infected and 5 μL of uninfected saliva. The inset shows the resonator frequency shift with respect to the plain graphene. At about 16.4183 GHZ, the uninfected saliva increased the resonant frequency, while the infected saliva reduced it. If the graphene layer exchanges electrons with the saliva through hybrid x bonds, the change in the resonant frequency indicates that the graphene layer donated electrons to the infected saliva, while the graphene layer accepted electrons from the uninfected saliva.

FIG. 8B shows the resonant frequency shift (MHZ) as a function of the saliva volume (μL) deposited on the paper filter in the 16.4 GHz F-P resonator with the paper filter sensor. The resonant frequency shift as a function of the saliva volume was negative in both infected and uninfected saliva cases when the paper filter was used.

FIG. 8C shows the S21 spectrum (dB) of the graphene layer between 0.75 GHZ and 0.83 GHZ. These spectra were normalized to the S21 spectrum of the plain graphene. The spectrum of the infected saliva (5 μL) is different than that of the uninfected saliva (5 μL) between 0.81-0.815 THz, as shown by the arrow.

The paper filter F-P sensor sensitivity using 16.4 GHZ F-B sensor, defined as Δf₀/δm was around 200 kHz/ng for high contrast conducting particles such as gold nanoparticles. The paper filter F-P sensor sensitivity was 500 kHz/mg for dielectric (polyester) micro-particles, and the paper filter F-P sensor sensitivity was around 800 KHz/1000 COVID-19 for viruses in saliva. The sensitivity using single atomic layer graphene at 16.4 GHz was 508 KHz/1000 COVID-19.

### Example 2: Voltage/Current, Capacitance, and Conductance of Viral Infected and Uninfected Saliva Coated with Aptamers or Uncoated

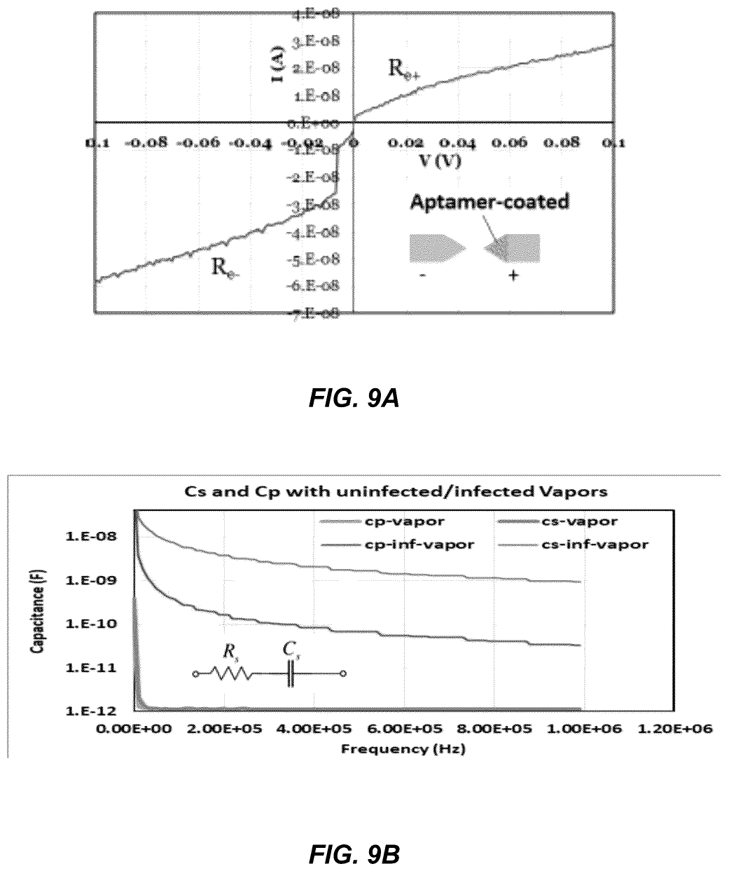

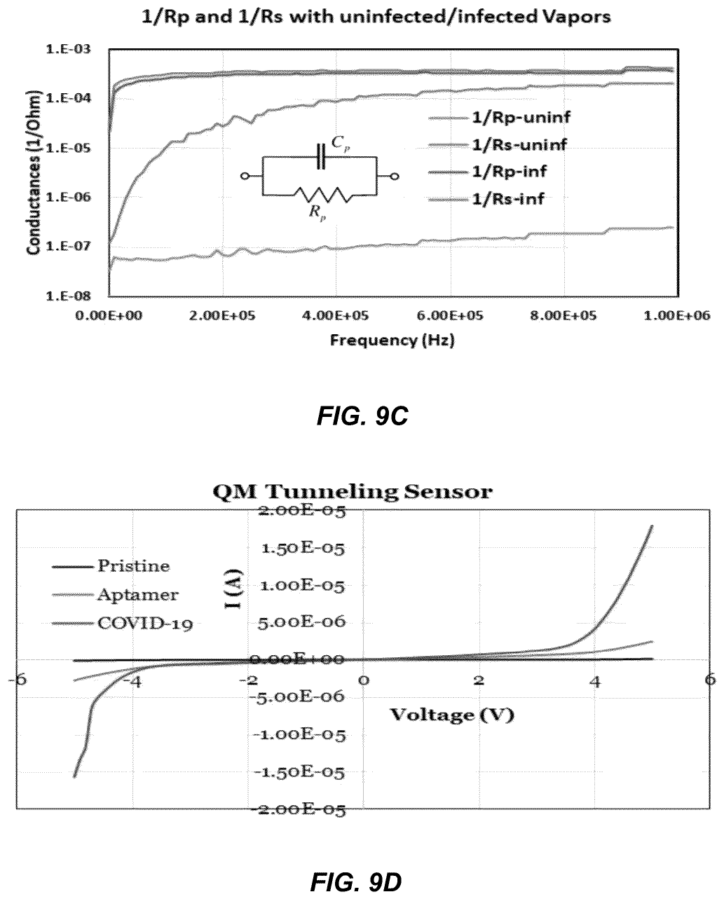

FIGS. 9A to 9D show responses of airborne particle sensors without aptamer, with aptamer alone, and with aptamer and bound virus. FIG. 9A shows the voltage/current (VI) graph of an aptamer-coated particle sensor and the measured resistance.

FIG. 9B shows the capacitance for the sample in infected vapor (Cₛ-inf-vapor) which was greater than the capacitance cp of the sample with aptamer in infected vapor. The non-infected samples were always less than the infected samples.

FIG. 9C shows the conductance for the infected sample in vapor (1/Rs-inf) which is similar to the conductance for the infected sample with the aptamer in vapor (1/Rp-inf). In contrast, the conductance for the uninfected sample (1/Rs-uninf) is much greater than the conductance for the uninfected sample with aptamer (1/Rp-uninf). Therefore, an aptamer-coated sample provided increased contrast between infected and uninfected samples.

FIG. 9D shows the IV graph for a pristine sample, an aptamer-coated sample, and a COVID-19 infected sample. The resistance for the COVID-19 sample varies greatly from the pristine and aptamer-coated samples as the voltage deviates from 0.

### Example 3: E-Field at 10 GHz at Different Phase Angles

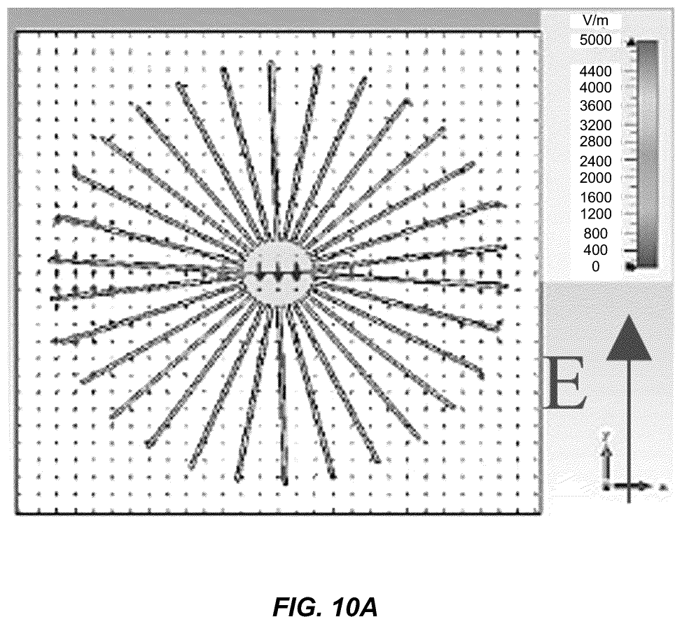

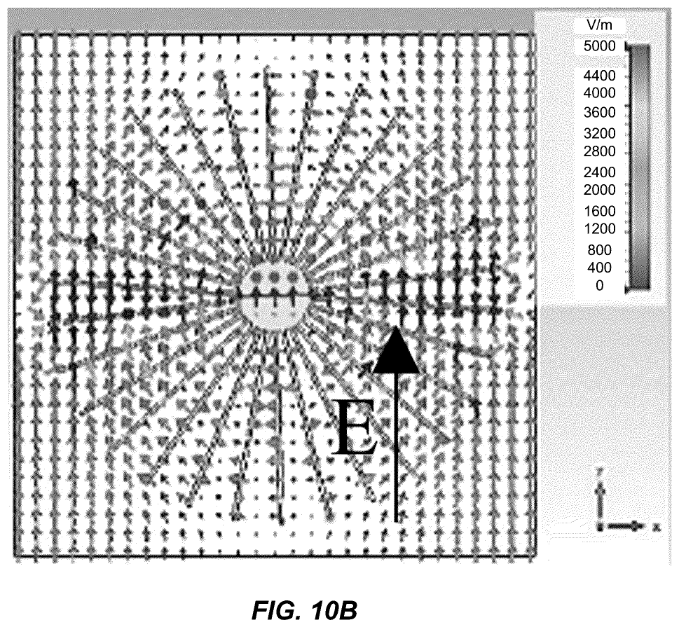

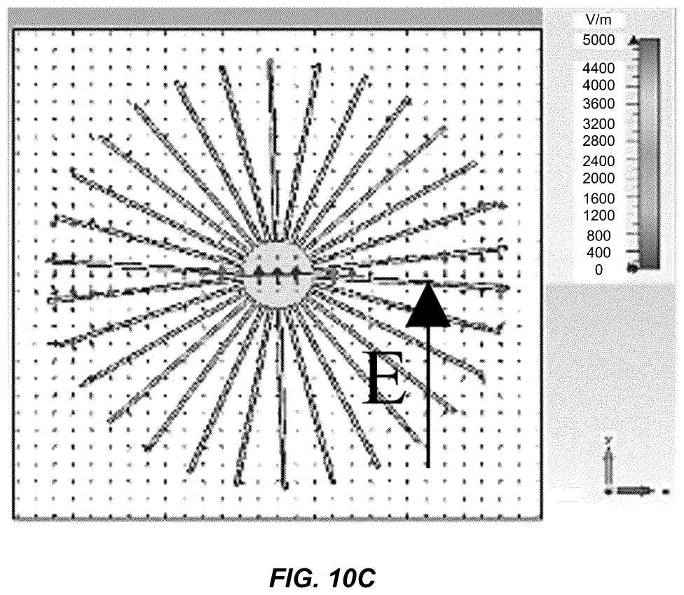

To understand the operation of the MTM sensor and gain insight into its behavior at resonances, the electric-field (E-field) was simulated at 10 GHZ with a vertically polarized E-field, as shown in FIGS. 10A to 10C. The E-field is shown at 10 GHz at different phase angles. FIG. 10A Phase=( ), FIG. 10B, Phase=90°, FIG. 10C Phase=180°. The incident field was vertically polarized. At 10 GHZ, the gap region facilitated a large E-field that could be used for sensitively detecting a sample.

### Example 4: Simulated E-field Components

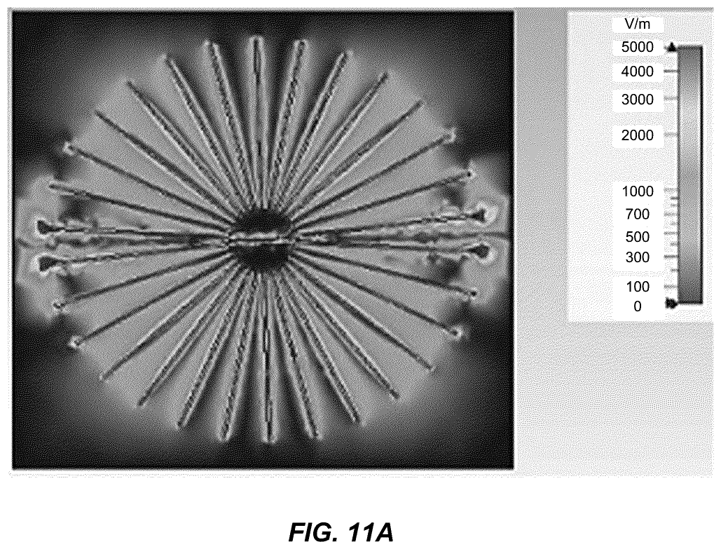

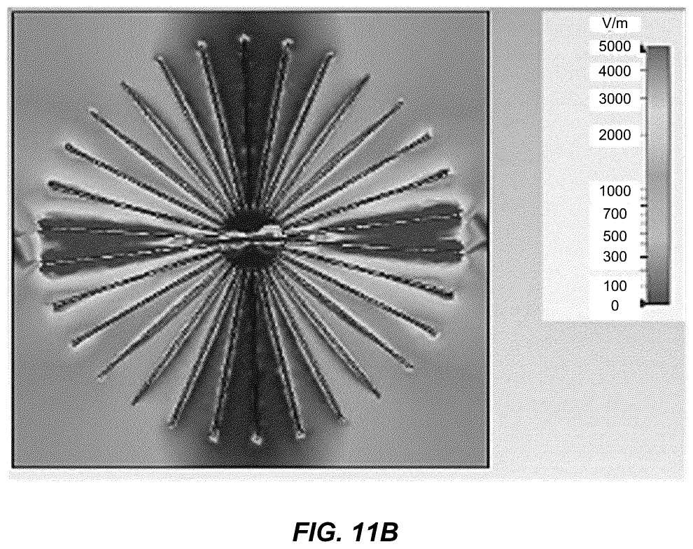

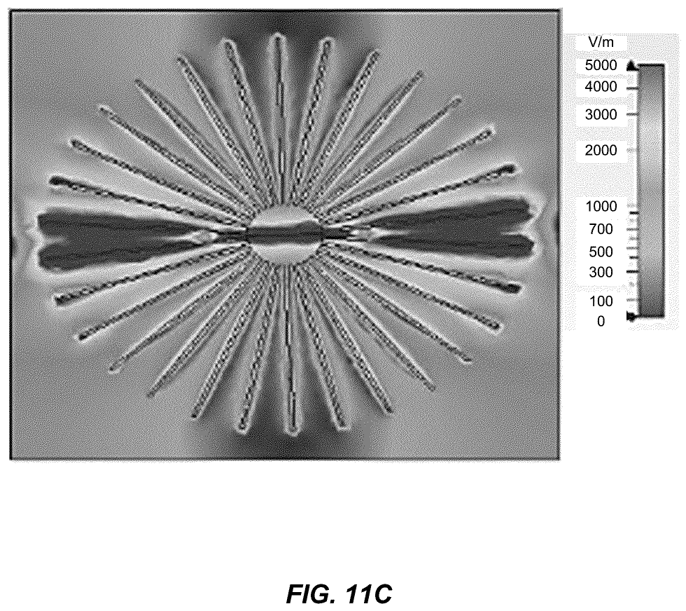

Other components of the E-fields in the sensor were also simulated and are shown in FIGS. 11A to 11C. The simulated E-field components in the spoke structure were simulated for the X-components, as shown in FIG. 11A, for the Y components, as shown in FIG. 11B, and for the combined E-field magnitude, as shown in FIG. 11C. The incident E field was vertical (Y). The E-field was polarized along the Y axis and coupled strongly with the vertical spokes setting up currents in the vertical spokes. The horizontal spokes did not couple with the incident vertically polarized E-fields. The simulation shows that these horizontal spokes contributed to the capacitive central section, as shown in FIG. 11B. Microwave signals with arbitrary or random E-field polarizations were intercepted with spokes at corresponding angles in the sensor. The interaction between the microwave signal and the sample (nanoparticles, saliva, etc.) was enhanced by the spoke structure. The electromagnetic properties of different materials in the gap region affected the MTM impedance differently, which facilitated the detection of the different materials by using reflection coefficient measurements.

### Example 5: S11 Spectra for Water and Saliva

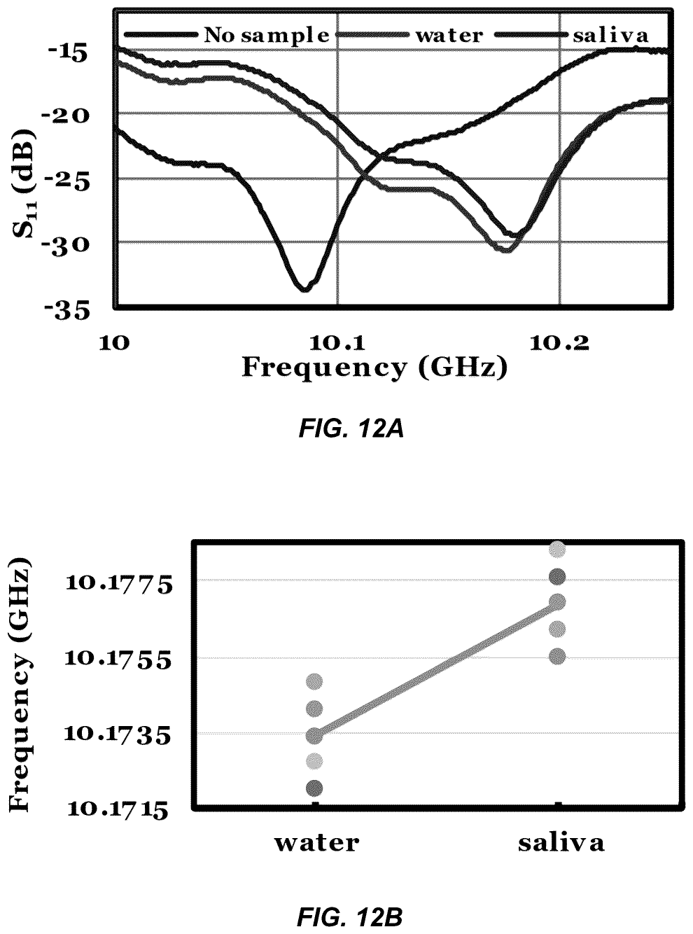

SARS-COV-2 Samples: 1 μL of different saliva samples were positioned in the sensor gap region: the tests were repeated five times for each sample. FIG. 12A shows the S11 spectra for 1 μL water and saliva sample placed in the gap. FIG. 12B shows the frequency spread at resonance for 1 μL of water and saliva samples. 1 μL was detectable and was the smallest sample size with ˜1000 viruses.

The shift in S11 spectra in FIG. 12A resulted from the slight difference between the dielectric constants of water and saliva. A healthy person's saliva has a relative average dielectric constant of ˜76 compared to the dielectric constant of ˜78 for water. So, the saliva has lower capacitance compared to water. As a result, the resonance frequency increased for saliva samples. FIG. 12B 8 shows the results of 5 different tests.

### Example 6: S11 Spectra for CoV-2 Infected Samples

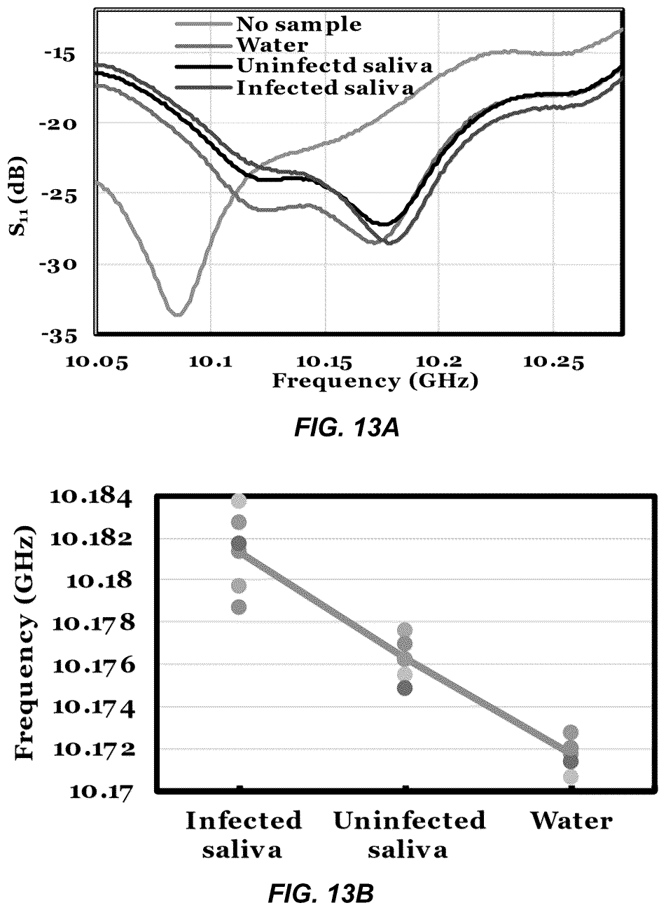

As shown in FIGS. 13A and 13B, 1 μL of SARS-COV-2 infected saliva was tested and compared to water, an uninfected saliva sample, and no sample. When compared to the S11 responses of deionized water, and uninfected saliva, there was a shift to the right in the resonance frequency, as shown in FIG. 13A. The frequency spread and average frequency response using 5 samples are shown in FIG. 13B. The increase in resonance frequency indicates a further decrease in the relative dielectric constant due to the presence of SARS COV-2 in the saliva. Both samples were 98-99% water and the shift in the resonant frequency was expected to be relatively small. Infected saliva lowered the resonance frequency by about 1.1 MHz.

### Example 7: S11 Spectra for Dielectric Samples

Dust and soot particles were deposited on the MTM sensors. These particulates can include carbon, silica, iron, or other oxides and salts particles. The electromagnetic properties of these particles can range from being purely dielectric in silica to semiconducting in soot to conducting and magnetic in iron and iron oxides.

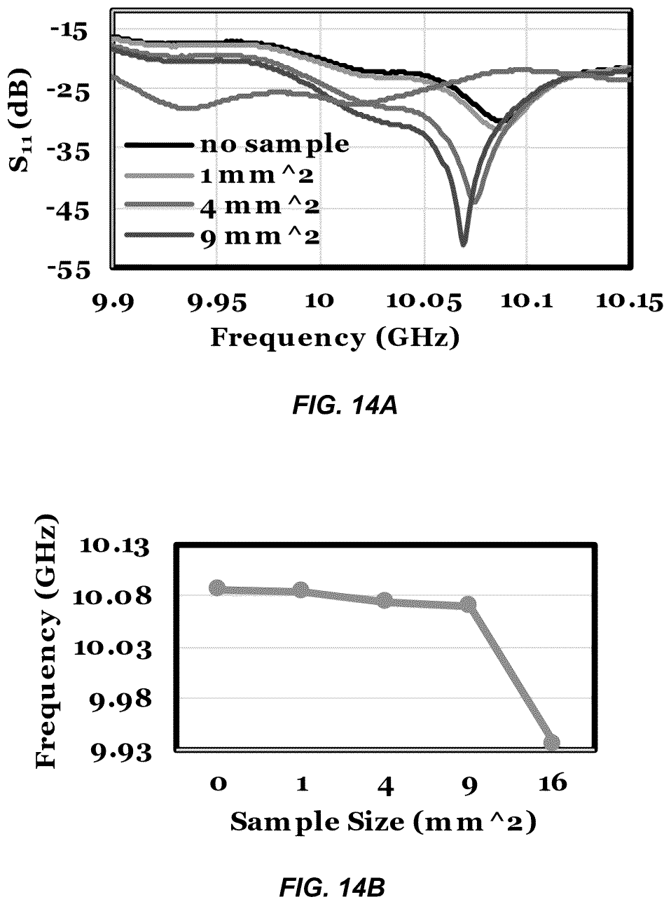

As shown in FIGS. 14A and 14B, a dielectric (duroid) sample with εᵣ=5.84 and regular paper samples with εᵣ˜1.8 were used. The S11 spectra results for duroid samples are shown in FIG. 14A, and the results for the resonance frequency as a function of duroid samples with different sizes are shown in FIG. 14B. The resonance frequency decreased as the gap's dielectric load increased until the dielectric sample beame relatively large (e.g., 3×3 mm²) which affected the Fabry-Perot resonances generated between the horn antenna and the MTM sensor. The average sensitivity of the sensor for duroid was about 3.23 MHz/mm³. The average sensitivity of the sensor for detecting paper was about 1.06 MHz/mm³.

### Example 8: S11 Spectra and Sensitivity for Iron Samples

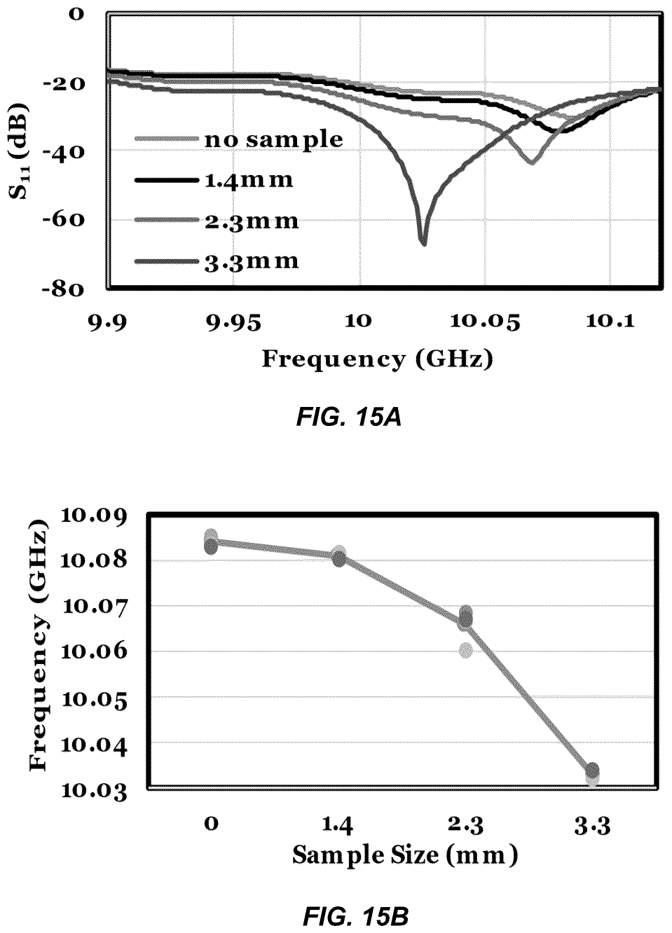

FIG. 15A to 15B

As shown in FIG. 15A and FIG. 15B, the S11 spectra with different lengths of a 1 mm diameter cylindrical iron sample showed that the average sensitivity was about 16.24 MHz/mm³.

### Example 9: Uniform Substate Detectability Using X-Band

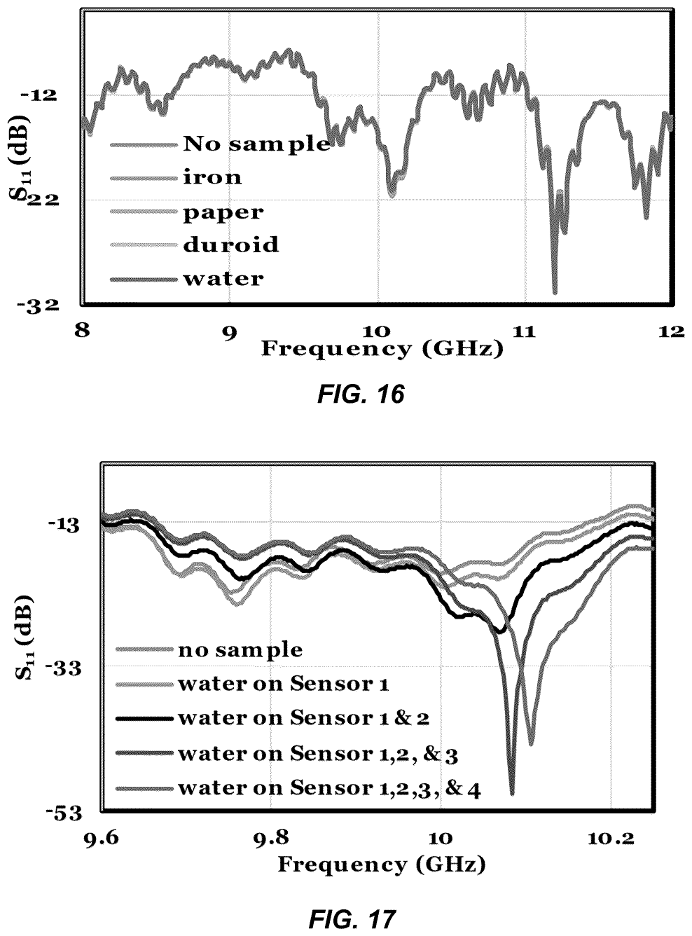

As shown in FIG. 16, the MTM sensor was replaced with a uniform dielectric substrate of the same dimensions to perform control experiments. FIG. 16 shows the S11 spectra for various samples (no sample, iron, paper, duroid, and water) placed on a dielectric substrate with εᵣ=3.43 for the X-Band frequencies of 8 GHz to 12 GHz. For these samples the spectra were superimposed because the small size of the samples did not allow for distinguishing the different sample types. However, when these samples were tested with the MTM sensor, the results provided allowed for distinguishing the spectra for different particle types. Consequently, the various samples could not be detected and differentiated when the uniform substrate was used. But when the MTM sensor structure was used, the samples had dimensions much smaller than (1/30) compared to the microwave wavelength of about ˜3 cm at 10 GHz, but different sample types were detectable and distinguishable.

### Example 10: S11 Spectra for Sensor Arrays

As shown in FIG. 17, to cover large areas for environmental sensing, an array of MTM sensors can be used as schematically shown in FIGS. 5A and 5B. The S11 spectra of each sensor of a 4 MTM sensor array was measured with and without a 1 μL water droplet. The shift in S11 frequency was largest when all four MTM sensors had water droplets. The location of the MTM sensor in the array also affected the S11 spectrum and could be used for multiplexing.

### Example 11: Sensitivity and Detection Time for Different Detection Methods

The resonance frequency of the MTM structure near ƒ₀=10 GHz was used as the sensor signal. The ƒ₀ was measured with different amounts of materials and used to calculate the sensor's sensitivity. The sensitivity (S) is S=Δf/d, where d is the physical dimension or volume of the material and Δf is the change in the sensor resonance frequency. Calibration curves (ƒ₀ versus the amount of material) were used to quantify the amount of substance that is detected by the sensor. Table I shows a comparison between the sensitivity of the MTM sensor disclosed herein and other sensor types. The sensor tested used about 10 GHz electromagnetic properties of the SARS-COV-2 viral particles that are different than other particles we examined. For enhanced specificity, the sensor surface can be coated with aptamers designed to bind with the spiking proteins of the target particle (e.g., SARS-COV-2).

TABLE I
Comparison between some recent SARS COV-2 detection
methods and the MTM microwave method disclosed herein

| | | | | Microwave |
|---|---|---|---|---|
| | | | Terahertz | sensor |
| Detection | | Electro- | plasmonic | disclosed |
| Method | Chemical | chemical | meta sensor | herein |
| Sensitivity | 1 ng/mL | 0.074 | 4.2 | 94.6 |
| | SARS-CoV-2 | fg/mL of | fM spike | MHz/1 μL |
| | antibodies | saliva | proteins | of saliva |
| Detection | 30 minutes | 100 ms | 80 minutes | Real time |
| time | | | | |

### Example 12: Summary

Therefore, it has been demonstrated that MTM sensors can detect small amounts of materials and objects (λ/30) using X-band microwave signals. The MTM sensors have a sensitivity in detecting SARS-COV-2 infected and uninfected human saliva of 94.6 MHz/1 μL and 89.5 MHz/1 μL, respectively. For dielectric samples (duroid) the sensitivity was about 3.23 MHz/mm³ and for metallic samples the sensitivity was about 16.25 MHz/mm³. An MTM sensor array was also interrogated for applications in sensing particles spread over large surfaces. The data presented here shows the feasibility of using an MTM-based particle sensor.

Reference throughout this specification to “an example” means that a particular feature, structure, or characteristic described in connection with the example is included in at least one embodiment of the present invention. Thus, appearances of the phrases “in an example” in various places throughout this specification are not necessarily all referring to the same embodiment.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. An airborne particle sensor for detecting a target particle, comprising:
a field generator source configured to emit an incident electromagnetic field;
a resonator having a focusing structure configured to focus the incident electromagnetic field in a gap region that is configured to accept the target particle; and
a receiver configured to detect a resonant signal from the resonator, wherein the resonant signal shifts due to presence of the target particle in the gap region.

2. The airborne particle sensor of claim 1, wherein the resonator comprises X-band metamaterials having a selected conductivity, a selected capacitance, or a combination thereof.

3. The airborne particle sensor of claim 1, wherein the resonator comprises at least one of: a Fabry-Perot (F-P) resonator, a coaxial resonator, a dielectric resonator, a crystal resonator, a ceramic resonator, a surface acoustic wave (SAW) resonator, and an yttrium iron garnet (YIG) resonator, and a combination thereof.

4. The airborne particle sensor of claim 1, wherein the focusing structure comprises one or more of:
a plurality of radial spokes connected to a central hub housing the gap region, wherein each radial spoke is flat or curved, and wherein the central hub is split or solid; or
a spiral structure comprising a central space housing the gap region, wherein the spiral structure is continuous or discrete; or
an hourglass structure comprising a central point housing the gap region: or
a flat structure comprising a plurality of distributed gap regions.

5. The airborne particle sensor of claim 1, wherein the gap region is a capacitive gap region having a capacitive gap of from 0.1 μm to about 1 mm.

6. The airborne particle sensor of claim 1, wherein the gap region is an inductive gap region.

7. The airborne particle sensor of claim 1, wherein a wavelength of the incident electromagnetic field is greater than a length of a sample captured by the resonator, wherein the sample includes the target particle.

8. The airborne particle sensor of claim 1, wherein the source emits the incident electromagnetic field having:
a microwave frequency ranging from about 5 GHz to about 20 GHz; or
a terahertz frequency ranging from about 0.75 GHz to about 0.83 GHz.

9. The airborne particle sensor of claim 1, wherein the incident electromagnetic field is an X-band electromagnetic field.

10. The airborne particle sensor of claim 1, wherein the source emits the incident electromagnetic field having a frequency capable of traversing a selected volume of from about 1 m to about 500 m without substantial attenuation.

11. The airborne particle sensor of claim 1, further comprising an inlet having a funnel structure configured to direct a sample having the target particle into the gap region.

12. The airborne particle sensor of claim 1, wherein the target particle comprises a pathogen that is a virus, a bacterium, or a fungus.

13. The airborne particle sensor of claim 1, wherein the airborne particle sensor has a sensitivity of:
from about 10 MHz/1 μL of pathogen sample to about 1000 MHz/1 μL of pathogen sample: or
from about 100 kHz/mm³ of dielectric sample to about 50 MHz/mm³ of dielectric sample; or
from about 1 MHz/mm³ of metal sample to about 100 MHz/mm³ of metal sample.

14. The airborne particle sensor of claim 1, further comprising a network analyzer operatively connected to the receiver to record responses, wherein the network analyzer is configured to calculate one or more of:
a transmission coefficient of the resonant signal; or
a resonator frequency shift when a target particle is accepted within the gap region; or
an amplitude modulation of the resonant signal; or
a phase shift of the resonant signal.

15. The airborne particle sensor of claim 1, further comprising:
a particle filter that is a porous layer positioned within 25% of a maximum of a standing wave of the incident electromagnetic field.

16. The airborne particle sensor of claim 15, wherein:
the particle filter is at least one of an atomic 2D layer, paper, and mesh; or
the particle filter is functionalized with an aptamer which selectively binds with a target particle.

17. The airborne particle sensor of claim 15, wherein the porous layer has a height ranging from about 0.01 mm to about 1 mm.

18. A method for sensing airborne particles, comprising:
emitting an incident electromagnetic field from a source;
focusing the incident electromagnetic field into a gap region of a resonator that is configured to accept a target particle;
detecting a resonant signal from the resonator.

19. The method of claim 18, further comprising:
calculating a resonator frequency shift when the target particle is accepted within the gap region: or
correlating the resonator frequency shift with a capture of the target particle in the gap region: or
calculating a transmission coefficient of the gap region: or
calculating an amplitude modulation of the resonant signal: or
calculating a phase shift of the resonant signal.

20. The method of claim 18, focusing the incident electromagnetic field into the gap region using a focusing structure comprising a metamaterial selected from one or more of:
a plurality of radial spokes connected to a central hub housing the gap region, wherein each radial spoke is flat or curved, and wherein the central hub is split or solid: or
a spiral structure comprising a central space housing the gap region, wherein the spiral structure is continuous or discrete: or
an hourglass structure comprising a central point housing the gap region: or
a flat structure comprising a plurality of distributed gap regions.

21. The method of claim 18, wherein a wavelength of the incident electromagnetic field is greater than a length of a sample captured by the resonator, wherein the sample includes the target particle.

22. The method of claim 18, further comprising:
emitting a microwave frequency ranging from about 5 GHz to about 20 GHz or a terahertz frequency ranging from about 0.75 GHz to about 0.83 GHz; or
emitting a microwave frequency that is an X-band electromagnetic field: or
emitting a frequency capable of traversing a selected volume with a threshold sensitivity.

23. The method of claim 18, further comprising:
directing a sample having the target particle into the gap region and ionizing the sample into separate particles.

24. The method of claim 18, further comprising:
positioning a particle filter at a standing wave electric field within the gap region:
accepting a target particle at particle filter oriented within the gap region: and
sending a response to a network analyzer operatively connected to the resonator.

25. An airborne particle sensing system, comprising:
a source configured to emit an incident electromagnetic field:
an array of airborne particle sensors housing in a ventilated structure, wherein each airborne particle sensors comprises a resonator having a focusing structure configured to focus the incident electromagnetic field in a gap region that is configured to accept a target particle; and
a receiver configured to detect the resonant signals from the array of airborne particle sensors.

26. The airborne particle sensing system of claim 25, wherein the array of airborne particle sensors is configured to sample a selected volume of air in a selected time.

27. The airborne particle sensing system of claim 25, wherein the ventilated structure is one or more of planar, three-dimensional, curved, flat, or a combination thereof.

28. The airborne particle sensing system of claim 25, wherein the source emits the incident electromagnetic field having:
a microwave frequency ranging from about 5 GHz to about 20 GHz; or
a terahertz frequency ranging from about 0.75 GHz to about 0.83 GHz: or
a microwave frequency that is an X-band electromagnetic field: or
a frequency capable of traversing a selected volume of from about 1 m to about 500 m without substantial attenuation.

29. The airborne particle sensing system of claim 25, further comprising an inlet having a funnel structure configured to:
direct one or more samples having the target particle into the gap regions of the array of airborne particle sensors and ionize the one or more samples into separate particles.

30. The airborne particle sensing system of claim 25, wherein the focusing structure comprises one or more of:
a plurality of radial spokes connected to a central hub housing the gap region, wherein each radial spoke is flat or curved, and wherein the central hub is split or solid; or
a spiral structure comprising a central space housing the gap region, wherein the spiral structure is continuous or discrete; or
an hourglass structure comprising a central point housing the gap region; or
a flat structure comprising a plurality of distributed gap regions.
